(19) United States
(12) Patent Application Publication
(10) Pub. No.: US 2026/0118260 A1
(43) Pub. Date: Apr. 30, 2026

(54) OPTICAL METHODS AND SYSTEMS FOR GLUCOSE MEASUREMENT

(71) Applicants: Anoushka S. NAIR, Hopkinton, MA (US); Dylan N. STRIEK, Hopkinton, MA (US)

(72) Inventors: Anoushka S. NAIR, Hopkinton, MA (US); Dylan N. STRIEK, Hopkinton, MA (US)

(21) Appl. No.: 19/065,476

(22) Filed: Feb. 27, 2025

Related U.S. Application Data
(60) Provisional application No. 63/714,394, filed on Oct. 31, 2024.

(51) Int. Cl. G01N 21/31; G01N 33/49

(52) U.S. Cl. CPC G01N 21/314; G01N 33/492; G01N 2021/3148

(57) ABSTRACT

The present disclosure teaches methods for optical estimation of blood glucose. In some embodiments, the disclosed methods involve estimating glucose levels based on absorbance detection of at least one electrolyte. Also provided are apparatuses for optical estimation of blood glucose, including instructions for use. In some embodiments, the apparatuses include light emitting units configured to emit light at specific wavelengths.

### CROSS-REFERENCE TO RELATED APPLICATIONS

This application claims priority to U.S. Provisional Application No. 63/714,394, filed on Oct. 31, 2024, which is hereby incorporated by reference in its entirety.

### FIELD

The present disclosure relates to methods and systems for glucose detection using optical processes. The methods and systems may employ devices configured for attachment to a portion of the body that transmit light at multiple wavelengths to detect the amount of one or more electrolytes in a body fluid such as blood, and determine a glucose concentration in the body fluid based on the detected amount of electrolytes.

### BACKGROUND

Detection of glucose in blood samples is a critical aspect of clinical diagnostics and blood sugar management. Technological advancements have expanded available methods for measuring blood sugar levels, allowing individuals with certain conditions, such as diabetes, to maintain target blood sugar ranges, prevent complications, and adjust treatment as needed. Traditional finger-prick blood glucometers continue to be widely used, and continuous glucose monitoring (CGM) systems facilitate real-time measurements through sensors inserted under the skin.

Emerging technologies, such as non-invasive optical spectroscopy and photoacoustic spectroscopy, show promise for pain-free glucose monitoring. While such methods offer the potential for non-invasive or minimally invasive monitoring, their efficacy is undermined by a variety of challenges, such as signal interference, calibration requirements, and achieving consistent accuracy across diverse patient populations. Other challenges may be related to complexity and cost. For example, there are several complex and expensive optical technologies under development that utilize NIR, MIR, Raman, and PA sensing, but these technologies to date have not been able to produce reliable glucose measurements. Accordingly, there exists a need to provide improved methods, systems, and devices for glucose detection.

### SUMMARY OF THE DISCLOSURE

In some embodiments, the present disclosure teaches methods, systems, and devices/apparatuses for the optical measurement of glucose within fluid samples, e.g., blood samples.

In some embodiments, the present disclosure teaches an apparatus for quantifying blood glucose, the apparatus comprising: a) a grip for releasably gripping a blood sample, the grip comprising first and second housings interconnected by a pivot configured to allow the first and second housings to pivot relative to one another to releasably grip the blood sample inserted between the first and second housings, the first and second housings being in electrical communication with each other; b) a light emitting unit comprised within the first housing, the light emitting unit positioned so as to direct light towards the second housing, through the blood sample gripped between the first and second housings, the light emitting unit comprising at least two of: i) a light source configured to emit light at 740-780 nm; ii) a light source configured to emit light at 580-620 nm; iii) a light source configured to emit light at 450-490 nm; and c) a light sensing unit comprised within the second housing, the light sensing unit comprising a photodetector configured to measure light transmittance from the light emitting unit, through the gripped blood sample.

In some embodiments, the apparatus further comprises a component for determining the distance between the light emitting unit and the light sensing unit.

In some embodiments, the pivot is configured to provide an angle between the first and second housings, thereby positioning the light emitting unit with respect to the light sensing unit.

In some embodiments, the light emitting unit comprises all three light sources recited in (b)(i)-(iii).

In some embodiments, the light emitting unit further comprises a light source configured to emit light at 625-675 nm.

In some embodiments, at least one of a plurality of light sources (e.g., three light sources) is a narrow-band emission light source. In some embodiments, each of the plurality of light sources (e.g., three light sources) is a narrow-band emission light source configured to emit light at 740-780 nm, 580-620 nm, or 450-490 nm. In some embodiments, the light source is a narrow-band light source configured to emit light at 625-675 nm. In some embodiments, at least one of the light sources is a light emitting diode (LED).

In some embodiments, the present disclosure teaches an apparatus according to earlier embodiments, wherein the light source (b)(i) is configured to emit light at about 766.5 nm; the light source (b)(ii) is configured to emit light at about 595 nm; and the light source (b)(iii) is configured to emit light at about 470 nm. In some embodiments, each of the light sources configured to emit light at about 766.5 nm, about 595 nm, or about 470 nm are light emitting diodes (LEDs).

In some embodiments, the apparatus comprises d) a power source. In some embodiments, the apparatus comprises e) a display.

In some embodiments, the light sensing unit comprises a plurality of photodetectors. In some embodiments, each photodetector of the plurality of photodetectors is arranged along the second housing at an angle of at least 5 degrees apart from each other, the angle measured from the light emitting unit, through the blood sample, and to the photodetector.

In some embodiments, the apparatus comprises a memory unit configured to store a first data matrix of measurements gathered by the photodetector.

In some embodiments, the apparatus comprises a processor configured to convert the measurements in the first data matrix into a glucose concentration, thereby quantifying glucose in the blood sample.

In some embodiments, the present disclosure teaches a method for quantifying a blood glucose level, the method comprising the steps of: a) measuring, via a photodetector, absorbance of blood irradiated with a light within at least two of the following wavelength ranges: i) 740-780 nm; ii) 580-620 nm; and iii) 450-490 nm; and b) determining glucose content estimates from the absorbance measurements from (a) by: i) dividing the absorbance measured in (a)(i) by (about 1.247×Lᵢ); ii) dividing the absorbance measured in (a)(ii) by (about 3.727×Lᵢᵢ); iii) dividing the absorbance measured in (a)(iii) by (about 3.533×Lᵢᵢᵢ); and iv) separately applying a regression model to the values of step (b)(i)-(iii), thereby producing a plurality of glucose content estimates; and c) reconciling the glucose content estimates from step (b), thereby quantifying blood glucose level of the blood; wherein L₍ᵢ₋ᵢᵢᵢ₎ is the distance the light travels though the blood in each of elements (a)(i)-(iii), respectively.

In some embodiments, the present disclosure teaches a method for quantifying a blood glucose level, the method comprising the steps of: a) measuring, via a photodetector, absorbance of blood irradiated with a light within at least two of the following wavelength ranges: i) 740-780 nm; ii) 580-620 nm; and iii) 450-490 nm; and b) determining glucose content estimates from the absorbance measurements from (a) by applying Beer's law to: i) absorbance measured in (a)(i) for potassium; ii) absorbance measured in (a)(ii) for phosphate; iii) absorbance measured in (a)(iii) for sodium; and c) applying a regression model to the values of step (b)(i)-(iii), thereby producing a plurality of glucose content estimates; and d) reconciling the glucose content estimates from step (c), thereby quantifying the blood glucose level.

In some embodiments, the method comprises irradiating the blood sample with light having a wavelength of about 766.5 nm for a(i), about 595 nm for a(ii), and about 470 nm for a(iii), if measured.

In some embodiments, the method, at step (c) and/or (d) comprises executing a neural network or machine learning algorithm.

### BRIEF DESCRIPTION OF THE DRAWINGS

FIG. 1A is a graph (standard curve) showing the absorbance of different amounts (mol) of potassium (K⁺) upon exposure to light wavelengths of 760.0 nm, 766.5 nm, and 770.0 nm.

FIG. 1B is a graph (standard curve) showing the absorbance of different amounts (mol) of sodium (Na⁺) upon exposure to light wavelengths of 595 nm, 600 nm, and 605 nm.

FIG. 1C is a graph (standard curve) showing the absorbance of different amounts (mol) of phosphate (PO₄³⁻) upon exposure to light wavelengths of 460.0 nm, 465 nm, and 470 nm.

FIG. 2 is a bar graph showing worm length and their frequency.

FIG. 3A is a graph showing the absorbance of K⁺ (766.5 nm), Na⁺ (595 nm), and PO₄³⁻ (470 nm) plotted against the glucose concentration of hemolymph isolated in worms from the control group (non-sugar diet). Regression curves shown for determination of a glucose modification factor.

FIG. 3B is a graph showing a plot of electrolyte levels (M) and corresponding glucose levels (M) from control group worms (non-sugar diet), obtained through interpolation of previously determined standard curves. Regression curves are also shown.

FIG. 4 is a bar chart of glucose levels (mg/dL) detected from the hemolymph of worms fed high-sugar (1:9 sugar:food ratio) and low-sugar diets 1:12 (sugar:food ratio).

FIG. 5A is a graph showing electrolyte absorbance values (A) of electrolytes K⁺ (766.5 nm), Na⁺ (595 nm), and PO₄³⁻ (470 nm) plotted against glucose concentration (mg/dL) obtained from the hemolymph of worms fed a low-sugar diet (1:12 sugar:food ratio). Regression curves shown for determination of a glucose modification factor.

FIG. 5B is a graph showing the molarity (M) of electrolytes K⁺, Na⁺, and PO₄³⁻ plotted against glucose molarity (M) obtained from the hemolymph of worms fed a low-sugar diet (1:12 sugar:food ratio). Regression lines are also shown.

FIG. 6A is a graph showing absorbance values (A) of electrolytes K⁺ (766.5 nm), Na⁺ (595 nm), and PO₄³⁻ (470 nm) plotted against glucose concentration (mg/dL) obtained from the hemolymph of worms fed a low-sugar diet (1:9 sugar:food ratio). Regression lines shown for determination of a glucose modification factor.

FIG. 6B is a graph showing the molarity (M) of electrolytes K⁺ (766.5 nm), Na⁺ (595 nm), and PO₄³⁻ (470 nm) plotted against glucose molarity (M) obtained from the hemolymph of worms fed a low-sugar diet (1:9 sugar:food ratio). Regression lines are also shown.

FIG. 7 is a graph showing the combined data for absorbance (A) of electrolytes K⁺ (766.5 nm), Na⁺ (595 nm), and PO₄³⁻ (470 nm) plotted against glucose concentration (mg/dL). Regression lines shown for determination of a glucose modification factor.

FIG. 8 is a schematic diagram of circuitry for the exemplary devices shown in FIGS. 9A and 9B. The schematic diagram depicts circuitry between an LED array configured to emit three wavelengths of light, a photodiode sensor, a microcontroller, and a power supply.

FIGS. 9A and 9B are diagrams of exemplary devices for estimating glucose levels from electrolyte absorption.

FIG. 10A shows another exemplary device for estimating glucose levels, where the device includes a battery holder, an OLED (organic light-emitting diode) display, and a clamp.

FIG. 10B shows the electronics cover, photodetector (photodiode), battery clip, and an array of LED bulbs for the device shown in FIG. 10A.

FIG. 11 is a schematic diagram of circuitry for another exemplary device for estimating glucose levels from electrolyte absorption.

FIG. 12 illustrates an exemplary approach for measuring absorption spectra by radiating light from a beam source through a sample to a detector, including the steps of emission, absorption, transmission, and detection.

FIGS. 13A and 13B are expanded views of the first and second housings, respectively, of the device shown in FIG. 9B. More specifically, the interior surface of the first and second housings is depicted.

### DETAILED DESCRIPTION

### Definitions

In the description and tables which follow, a number of terms are used. In order to provide a clear and consistent understanding of the specification and claims, including the scope to be given such terms, the following definitions are provided.

The term “a” or “an” refers to one or more of that entity; for example, “a LED” refers to one or more LEDs or at least one LED. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

The term “about” when immediately preceding a numerical value means a range (e.g., plus or minus 10% of that value). For example, “about 50” can mean 45 to 55, “about 25,000” can mean 22,500 to 27,500, etc., unless such an interpretation would result in a value above or below range of possible values, such as below 0% or above 100% of a possible value. Furthermore, the phrases “less than about” a value or “greater than about” a value should be understood in view of the definition of the term “about” provided herein, as applied to any recited endpoint. Similarly, the term “about” when preceding a series of numerical values or a range of values (e.g., “about 10, 20, 30” or “about 10-30”) refers, respectively to all values in the series, or the endpoints of the range. Unless otherwise indicated, it is to be understood that all numbers expressing quantities, ratios, and numerical properties of ingredients, reaction conditions, and so forth, used in the specification and claims are contemplated to be able to be modified in all instances by the term “about”.

The term “approximately” when immediately preceding a numerical value means a range (e.g., plus or minus 5% of that value). For example, “approximately 50” can mean 47.5 to 52.5, “approximately 25,000” can mean 23,750 to 26,250, etc., unless such an interpretation would result in a value above or below range of possible values, such as below 0% or above 100% of a possible value.

The term “including all ranges and sub-ranges therein” or equivalents, are used herein to denote the intention that disclosure of any range or series of possible values, inherently also discloses all ranges and subranges encompassed by the highest and lowest values disclosed. This term includes the entire range from highest to lowest disclosed values, as well as subranges from any two or more disclosed points. This term is also intended to disclose any subranges encompassed anywhere within the highest and lowest disclosed values, including between two points that are explicitly recited in the document, up to one decimal point. Thus, disclosure of values 0, 5, 10, 15, 20, including all ranges and subranges therebetween, should be interpreted as also encompassing a range from 0-20, a range from 0-5 or 5-15, as well as a range from 2-16, or 3.1 to 19.8, etc.

Unless otherwise indicated, it is to be understood that all numbers expressing quantities, ratios, and numerical properties of ingredients, reaction conditions, and so forth, used in the specification are contemplated to be able to be modified in all instances by the term “including all ranges and sub-ranges therein.”

### Overview

There is a great interest in developing optical methods for sensing glucose in blood, particularly for applications in continuous glucose monitoring. Current approaches for optically detecting glucose in blood, however, face several challenges related to complexity and cost. For example, and as previously mentioned, there are several complex and expensive optical technologies under development that utilize NIR, MTR, Raman, and PA sensing, but these technologies to date have not been able to produce reliable glucose measurements. Accordingly, described herein are methods and systems for optically estimating the glucose content of fluid samples such as blood, plasma, urine, cerebrospinal fluid, pleural fluid, ascitic fluid, and sweat. In some embodiments, the methods and systems disclosed herein are used to measure the level of glucose in a blood sample. In some embodiments, the methods and systems disclosed herein are used to non-invasively estimate the glucose level of a blood sample, in vitro, or in vivo. In general, the methods and systems may include a grip device, clamp, clip, or other device having arms coupled to one another, which may be configured to optically detect one or more electrolytes (e.g., sodium, potassium, and/or phosphate) and estimate blood glucose content based on the detected amount of the one or more electrolytes.

### Glucose Estimation Through Electrolyte Measurements

As noted above, one of the challenges in developing optical measurements for blood glucose is that the interaction of glucose with light occurs in non-visible regions of the electromagnetic spectrum. Glucose has maximum absorption at a wavelength of 939 nm, 970 nm, and 1197 nm in higher overtone regions; a wavelength of 1408 nm, 1536 nm, 1688 nm, and 1925 nm in the first overtone region; and a wavelength of 2100 nm, 2261 nm, and 2326 nm in a combination region. This inconvenient absorbance spectrum of glucose made development of commercial-grade solutions impractical to date. See, e.g., Javid B, Fotouhi-Ghazvini F, Zakeri FS. Noninvasive Optical Diagnostic Techniques for Mobile Blood Glucose and Bilirubin Monitoring. J Med Signals Sens. 2018 July-September; 8(3):125-139.

In some embodiments, the present disclosure teaches methods for quantifying a blood glucose level, comprising the steps of a) measuring, via a photodetector, absorbance of blood irradiated with a light within at least two of the following wavelength ranges: i) 740-780 nm; ii) 580-620 nm; iii) 450-490 nm; and b) determining glucose content estimates from the absorbance measurements by (a) using the Beer-Lambert law and applying a regression model based on a previously determined relationship between absorbance at those wavelength ranges and blood glucose; and c) reconciling the glucose content estimates from step (b), thereby quantifying blood glucose level of the blood. Each of these aspects is explored in more detail, infra.

In some aspects, the present disclosure solves the challenges faced in the art by providing apparatuses and methods for estimating glucose levels from absorbance detection of one or more electrolytes associated with glucose in blood. The apparatuses and methods are thus based, at least in part, on applicant's recognition that measurements of selected electrolytes can be used or combined to obtain blood glucose level estimates.

In some embodiments, glucose levels are estimated from absorbance detection of sodium, potassium, chloride, magnesium, calcium, phosphate, bicarbonate, or combinations thereof. In some embodiments, glucose levels are estimated from absorbance detection of at least one, two, three, four, five, or six electrolytes. In some embodiments, glucose levels are estimated from absorbance detection of a total of one, two, three, four, five, six, or seven electrolytes. For example, in some embodiments, glucose levels are estimated from absorbance measurements of sodium, potassium, and phosphate ions. In some embodiments, glucose levels are estimated from absorbance measurements of sodium, and potassium.

These electrolytes or ions (sodium, potassium, phosphate) were selected based on their ubiquity within blood and association with glucose. The identified electrolytes are essential for a variety of cellular processes, including maintaining electrical neutrality in cells, as well as generating and conducting action potentials in nerves and muscles, and are therefore well-represented within blood. For example, in some embodiments, glucose levels are estimated from absorbance measurements of sodium, potassium, and phosphate ions. In some embodiments, glucose levels are estimated from absorbance measurements of sodium, and potassium. Each of these is discussed in further detail below.

i. Sodium

Sodium (Na⁺) is an osmotically active cation that is important for maintaining extracellular fluid volume and regulating cellular membrane potential. The regulation of sodium levels in the body is primarily managed by the kidneys through a complex process of reabsorption. See, e.g., Ferrannini. Cell Metab. 2017 Jul. 5; 26(1):27-38.

Sodium levels have a complex relationship with excess blood sugar. For example, hyperglycemia can cause a decrease in serum sodium concentration. High blood sugar causes water to shift from inside cells to the extracellular space, diluting the amount of sodium in the blood. This can lead to hyponatremia in patients with severe hyperglycemia. See, e.g., Chuang et al., J Chin Med Assoc. 2020 September; 83(9): 845-851.

ii. Potassium

Potassium (K⁺) is an important intracellular cation involved in maintaining cellular homeostasis and physiological functions. The sodium-potassium adenosine triphosphatase (Na+/K+-ATPase) pump is important in regulating the balance between extracellular sodium and intracellular potassium concentrations. This transmembrane protein actively transports sodium ions out of the cell while simultaneously importing potassium ions, maintaining the electrochemical gradient essential for various cellular processes. Renal regulation of potassium homeostasis is a complex process involving multiple nephron segments. Conversely, hyperkalemia, where serum potassium exceeds 5.5 mmol/L, poses a risk for cardiac arrhythmias and may manifest as muscle cramps, weakness, or in extreme cases, rhabdomyolysis with subsequent myoglobinuria.

Potassium levels in blood have been associated with diabetes and blood glucose levels. For example, insulin, a hormone released in response to elevated blood glucose, influences potassium balance. When insulin is administered or released, it promotes the uptake of glucose into cells and simultaneously facilitates the entry of potassium into cells. Therefore, conditions associated with insulin deficiency, like diabetic ketoacidosis, can lead to elevated blood levels of both glucose and potassium. See, e.g., Pakistan Journal of Medical Sciences, vol. 35, no. 3, 22 May 2019. See also PLoS One. 2016 Jun. 9; 11(6):e0157252. doi: 10.1371/journal.pone.0157252. PMID: 27280455; PMCID: PMC4900670.

iii. Phosphate

Phosphate (PO₄³⁻) is an essential mineral that's involved in many biochemical processes, including ATP formation, cell membrane formation, and DNA and RNA synthesis. Phosphate is also involved in energy balance. When cells need energy, ATPase enzymes cleave the terminal phosphate of adenosine triphosphate (ATP) to form adenosine diphosphate (ADP) and inorganic phosphate. Thus, phosphate levels in the blood correlate to energy usage.

Additional research suggests that disturbances in phosphate levels may be associated with insulin resistance and impaired glucose metabolism. However, the exact mechanisms and relationships are complex and may vary based on specific conditions. January 2012 Journal of Bone and Mineral Metabolism 30(1):10-8; see also Nutrients. 2022 Oct. 25; 14(21):4477.

### Absorbance Detection of Electrolytes

As noted above, previous efforts at developing optical methods for glucose detection were stymied by glucose's inconvenient optical absorption spectrum. In contrast, the present disclosure teaches optical detection of electrolytes that can be used to estimate blood glucose content. These electrolytes were found to be detectable via optical absorption techniques at wavelengths that are achievable by less expensive equipment. Thus, in some embodiments, the present disclosure teaches measuring, via a photodetector, absorbance of blood irradiated with a light within at least two of the following wavelength ranges: i) 740-780 nm; ii) 580-620 nm; and iii) 450-490 nm, which each correspond to an electrolyte of interest (e.g., potassium, sodium, and phosphate, respectively).

In some embodiments, detection of these electrolytes is conducted via optical absorbance. Absorption spectroscopy is spectroscopy that involves techniques that measure the absorption of electromagnetic radiation, as a function of frequency or wavelength, due to its interaction with a sample. The sample absorbs energy, i.e., photons, from the radiating field. The intensity of the absorption varies as a function of frequency, and this variation is the absorption spectrum. Absorption spectroscopy is performed across the electromagnetic spectrum.

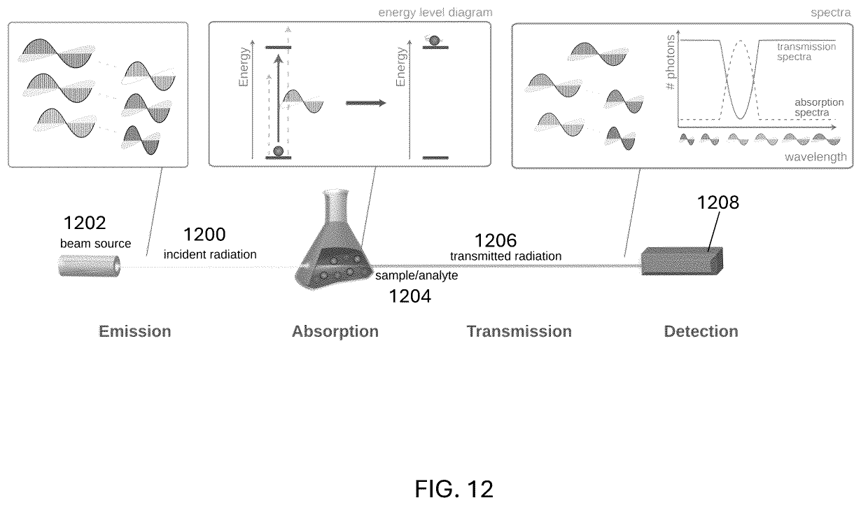

Absorption spectroscopy is employed as an analytical chemistry tool to determine the presence of a particular substance in a sample and, in many cases, to quantify the amount of the substance present. There is a wide range of experimental approaches for measuring absorption spectra. For example, as illustrated in FIG. 12, a beam of radiation (e.g., light at a predetermined wavelength; incident radiation (1200)) generated by a beam source (1202) can be emitted towards a sample (1204) and the radiation (1206) transmitted through the sample detected by photodetector (1208). The transmitted energy can be used to calculate the absorption.

A material's absorption spectrum is generally the fraction of incident radiation absorbed by the material over a range of frequencies of electromagnetic radiation. The absorption spectrum is primarily determined by the atomic and molecular composition of the material. Radiation is more likely to be absorbed at frequencies that match the energy difference between two quantum mechanical states of the molecules.

Absorption and transmission spectra are related concepts, such that absorption and transmittance values can be converted to each other mathematical transformation. A transmission spectrum will have its maximum intensities at wavelengths where the absorption is weakest because more light is transmitted through the sample. An absorption spectrum will have its maximum intensities at wavelengths where the absorption is strongest. In some embodiments, transformation between the two values is governed by the formula A=log₁₀(1/T), where A is absorbance, and T=transmittance.

In some embodiments, the instant disclosure teaches obtaining concentrations of one or more electrolytes in a blood sample to estimate a blood glucose level. Thus, aspects of the disclosure relate to converting absorption measurements to corresponding concentration values. In some embodiments, electrolyte concentration is based on Beer-Lambert principles.

Beer-Lambert law, commonly called Beer's law, states that a beam of visible light passing through a chemical solution of fixed geometry experiences absorption proportional to the solute concentration. The simplest formulation of Beer's law is embodied in the following formula: A=E×L×C, where A is absorbance at the appropriate wavelength for the detected molecule of interest, L is the length of the beam passing through the medium, and C is the molar concentration of the molecule of interest. In this formula, E is the extinction coefficient governing the relationship between absorbance and concentration at a particular wavelength.

The present disclosure describes the exploration and identification of wavelengths useful for detecting each electrolyte of interest, and the corresponding calculation of the extinction coefficient for each electrolyte at the determined wavelength. See e.g., Example 1.

In some embodiments, the instant disclosure teaches optically measuring the concentration of potassium at a wavelength between about 740-780 nm, including all values and sub-ranges therein. For example, the concentration of potassium may be optically measured using light having a wavelength of about 740 nm, 740 nm, 741 nm, 742 nm, 743 nm, 744 nm, 745 nm, 746 nm, 747 nm, 748 nm, 749 nm, 750 nm, 751 nm, 752 nm, 753 nm, 754 nm, 755 nm, 756 nm, 757 nm, 758 nm, 759 nm, 760 nm, 761 nm, 762 nm, 763 nm, 764 nm, 765 nm, 766 nm, 766.5 nm, 767 nm, 768 nm, 769 nm, 770 nm, 771 nm, 772 nm, 773 nm, 774 nm, 775 nm, 776 nm, 777 nm, 778 nm, 779 nm, or 780 nm, including all ranges and subranges therebetween. In some embodiments, the concentration of potassium may be optically measured using light having a wavelength of about 766.5 nm.

In some embodiments, the instant disclosure teaches optically measuring the concentration of phosphate at a wavelength between about 450-490 nm, including all values and sub-ranges therein. For example, the concentration of phosphate may be optically measured using light having a wavelength of about 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, or 490 nm, including all ranges and subranges therebetween. In some embodiments, the concentration of phosphate may be optically measured using light having a wavelength of about 470 nm.

In some embodiments, the instant disclosure teaches optically measuring the concentration of sodium at a wavelength between about 580-620 nm, including all values and sub-ranges therein. For example, the concentration of sodium may be optically measured using light having a wavelength of about 580 nm, 581 nm, 582 nm, 583 nm, 584 nm, 585 nm, 586 nm, 587 nm, 588 nm, 589 nm, 590 nm, 591 nm, 592 nm, 593 nm, 594 nm, 595 nm, 596 nm, 597 nm, 598 nm, 599 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, or 620 nm, including all ranges and subranges therebetween. In some embodiments, the concentration of sodium may be optically measured using light having a wavelength of about 595 nm.

In some embodiments, the optically measured concentration of one or more electrolytes is based on a single optical measurement. In some embodiments, the electrolyte concentration is based on a plurality of measurements. In some embodiments, the plurality of measurements can be reconciled via well known error-correction techniques. For example, in some embodiments, the plurality of electrolyte measurements is averaged. In some embodiments the measurements are processed via a data validation and reconciliation model. In some embodiments, the values are filtered through gross error detection and elimination techniques, including chi square or individual test. In some embodiments, measured concentrations are also filtered based on pre-set value gates based on known biological ranges for the electrolyte and/or historical readings programmed into a processor or obtained from recent measurements. In some embodiments, the measured concentrations of an electrolyte are also compared against measurements taken by redundant light sources/sensors that may be included in a measuring device. Known value correlations between concentrations of each of the electrolytes may also be considered.

In some embodiments the concentration of each electrolyte of interest can be calculated based on the optical measurements. In some embodiments the calculations are according to the Beer-Lambert law. In some embodiments, the extinction coefficient for potassium is between 1 to 1.5, including all values and sub-ranges therein. For example, the extinction coefficient for potassium may be about 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5, including all ranges and subranges therebetween. In some embodiments, the extinction coefficient for potassium is about 1.247.

In some embodiments, the extinction coefficient for sodium is between 3.2 to 4, including all values and sub-ranges therein. For example, the extinction coefficient for sodium may be about 3.2, 3.21, 3.22, 3.23, 3.24, 3.25, 3.26, 3.27, 3.28, 3.29, 3.3, 3.31, 3.32, 3.33, 3.34, 3.35, 3.36, 3.37, 3.38, 3.39, 3.4, 3.41, 3.42, 3.43, 3.44, 3.45, 3.46, 3.47, 3.48, 3.49, 3.5, 3.51, 3.52, 3.53, 3.54, 3.55, 3.56, 3.57, 3.58, 3.59, 3.6, 3.61, 3.62, 3.63, 3.64, 3.65, 3.66, 3.67, 3.68, 3.69, 3.7, 3.71, 3.72, 3.73, 3.74, 3.75, 3.76, 3.77, 3.78, 3.79, 3.8, 3.81, 3.82, 3.83, 3.84, 3.85, 3.86, 3.87, 3.88, 3.89, 3.9, 3.91, 3.92, 3.93, 3.94, 3.95, 3.96, 3.97, 3.98, 3.99, or 4, including all ranges and subranges therebetween. In some embodiments, the extinction coefficient for sodium is about 3.727.

In some embodiments, the extinction coefficient for phosphate is between 3 to 4, including all values and sub-ranges therein. For example, the extinction coefficient for phosphate may be about 3, 3.01, 3.02, 3.03, 3.04, 3.05, 3.06, 3.07, 3.08, 3.09, 3.1, 3.11, 3.12, 3.13, 3.14, 3.15, 3.16, 3.17, 3.18, 3.19, 3.2, 3.21, 3.22, 3.23, 3.24, 3.25, 3.26, 3.27, 3.28, 3.29, 3.3, 3.31, 3.32, 3.33, 3.34, 3.35, 3.36, 3.37, 3.38, 3.39, 3.4, 3.41, 3.42, 3.43, 3.44, 3.45, 3.46, 3.47, 3.48, 3.49, 3.5, 3.51, 3.52, 3.53, 3.54, 3.55, 3.56, 3.57, 3.58, 3.59, 3.6, 3.61, 3.62, 3.63, 3.64, 3.65, 3.66, 3.67, 3.68, 3.69, 3.7, 3.71, 3.72, 3.73, 3.74, 3.75, 3.76, 3.77, 3.78, 3.79, 3.8, 3.81, 3.82, 3.83, 3.84, 3.85, 3.86, 3.87, 3.88, 3.89, 3.9, 3.91, 3.92, 3.93, 3.94, 3.95, 3.96, 3.97, 3.98, 3.99, or 4, including all ranges and subranges therebetween. In some embodiments, the extinction coefficient for sodium is about 3.533.

As noted above, the Beer-Lambert law also uses variable “L”, the length of light traveling through the fluid sample of interest (e.g., through blood). In some embodiments, L is a known value, such as the thickness of the blood volume within a sample (e.g., space within a cuvette). In some embodiments L is provided by an apparatus/device of the system (e.g., an apparatus/device configured to attach to a body portion of a user), which reports on the thickness of the sample. In some embodiments, L is reported by a hinge (e.g., a pivot) of the apparatus/device, as further described below, which is configured to calculate sample thickness.

In some embodiments, a photodetector disposed within the apparatus/device may be configured to determine L. For example, light having a wavelength of about 650 nm may be used to detect blood volume because it falls within the red light spectrum where hemoglobin, the protein in blood that carries oxygen, absorbs significantly, allowing for measurement of blood volume changes based on how much light is absorbed at this wavelength. This principle is often utilized in technologies like pulse oximeters, which use both red (around 660 nm) and infrared light to determine blood oxygen saturation.

In some embodiments, the instant disclosure teaches optically measuring blood volume with light having a wavelength between about 620-680 nm, including all values and sub-ranges therein. For example, blood volume may be optically measured using light at a wavelength of about 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 651 nm, 652 nm, 653 nm, 654 nm, 655 nm, 656 nm, 657 nm, 658 nm, 659 nm, 660 nm, 661 nm, 662 nm, 663 nm, 664 nm, 665 nm, 666 nm, 667 nm, 668 nm, 669 nm, 670 nm, 671 nm, 672 nm, 673 nm, 674 nm, 675 nm, 676 nm, 677 nm, 678 nm, 679 nm, or 680 nm, including all ranges and subranges therebetween. In some embodiments, the present disclosure teaches optically measuring blood volume with light at a wavelength of about 650 nm. This blood volume then informs the value of L in the calculation of electrolyte concentrations.

Glucose Concentration from Electrolyte Measurements

The methods and apparatuses described herein may be used to determine (e.g., by estimating) blood glucose content based on electrolyte concentrations in blood. Thus, in some embodiments, the present disclosure teaches methods and apparatuses for identifying and describing the relationship between glucose and the concentration of one or more electrolytes of interest (e.g., sodium, potassium, and/or phosphate). Specifically, the instant disclosure describes taking the absorbance values measured for various electrolytes and applying a regression model based on a previously determined relationship between absorbance at those wavelength ranges and blood glucose. In some embodiments, the present disclosure teaches correlation models that associate concentrations of potassium, sodium, and/or phosphate to blood glucose concentrations. Example 2 illustrates the correlation model creation.

In some embodiments the correlational models are generated using empirical data of known electrolyte and blood glucose models, and applying one or more regression model. Persons having skill in the art will be familiar with available regression models. In some embodiments, the regression model is selected form the group consisting of: linear regression, polynomial regression, elasticnet regression, stepwise regression, partial least squares regression, principal components regression, Poisson regression, logistic regression, lasso regression, nonlinear regression, support vector regression, quantile regression, random forest regression, ordinal regression, ridge regression, multiple regression simple regression, decision tree regression, Bayesian regression, stepwise regression. In some embodiments, the regression utilizes machine learning.

Thus, in some embodiments, the present disclosure teaches providing a plurality of known electrolyte concentrations with corresponding known blood glucose concentrations, and applying a regression technique to produce a finalized correlational regression model. In some embodiments, the regression is applied only to data points within a biologically-relevant range of glucose concentrations for the animal of interest. For example, in some embodiments, regression models are based on a blood glucose content ranging from about 10 mg/dL to about 500 mg/dL, including all values and sub-ranges therein. In some embodiments, the blood glucose content range from about 50 to about 350 mg/dL, including all values and sub-ranges therein. In some embodiments, the blood glucose content ranges from about 70 mg/dL to about 300 mg/dL. For example, the blood glucose content may be about 10 mg/dL, 11 mg/dL, 12 mg/dL, 13 mg/dL, 14 mg/dL, 15 mg/dL, 16 mg/dL, 17 mg/dL, 18 mg/dL, 19 mg/dL, 20 mg/dL, 21 mg/dL, 22 mg/dL, 23 mg/dL, 24 mg/dL, 25 mg/dL, 26 mg/dL, 27 mg/dL, 28 mg/dL, 29 mg/dL, 30 mg/dL, 31 mg/dL, 32 mg/dL, 33 mg/dL, 34 mg/dL, 35 mg/dL, 36 mg/dL, 37 mg/dL, 38 mg/dL, 39 mg/dL, 40 mg/dL, 41 mg/dL, 42 mg/dL, 43 mg/dL, 44 mg/dL, 45 mg/dL, 46 mg/dL, 47 mg/dL, 48 mg/dL, 49 mg/dL, 50 mg/dL, 51 mg/dL, 52 mg/dL, 53 mg/dL, 54 mg/dL, 55 mg/dL, 56 mg/dL, 57 mg/dL, 58 mg/dL, 59 mg/dL, 60 mg/dL, 61 mg/dL, 62 mg/dL, 63 mg/dL, 64 mg/dL, 65 mg/dL, 66 mg/dL, 67 mg/dL, 68 mg/dL, 69 mg/dL, 70 mg/dL, 71 mg/dL, 72 mg/dL, 73 mg/dL, 74 mg/dL, 75 mg/dL, 76 mg/dL, 77 mg/dL, 78 mg/dL, 79 mg/dL, 80 mg/dL, 81 mg/dL, 82 mg/dL, 83 mg/dL, 84 mg/dL, 85 mg/dL, 86 mg/dL, 87 mg/dL, 88 mg/dL, 89 mg/dL, 90 mg/dL, 91 mg/dL, 92 mg/dL, 93 mg/dL, 94 mg/dL, 95 mg/dL, 96 mg/dL, 97 mg/dL, 98 mg/dL, 99 mg/dL, 100 mg/dL, 101 mg/dL, 102 mg/dL, 103 mg/dL, 104 mg/dL, 105 mg/dL, 106 mg/dL, 107 mg/dL, 108 mg/dL, 109 mg/dL, 110 mg/dL, 111 mg/dL, 112 mg/dL, 113 mg/dL, 114 mg/dL, 115 mg/dL, 116 mg/dL, 117 mg/dL, 118 mg/dL, 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, 131 mg/dL, 132 mg/dL, 133 mg/dL, 134 mg/dL, 135 mg/dL, 136 mg/dL, 137 mg/dL, 138 mg/dL, 139 mg/dL, 140 mg/dL, 141 mg/dL, 142 mg/dL, 143 mg/dL, 144 mg/dL, 145 mg/dL, 146 mg/dL, 147 mg/dL, 148 mg/dL, 149 mg/dL, 150 mg/dL, 151 mg/dL, 152 mg/dL, 153 mg/dL, 154 mg/dL, 155 mg/dL, 156 mg/dL, 157 mg/dL, 158 mg/dL, 159 mg/dL, 160 mg/dL, 161 mg/dL, 162 mg/dL, 163 mg/dL, 164 mg/dL, 165 mg/dL, 166 mg/dL, 167 mg/dL, 168 mg/dL, 169 mg/dL, 170 mg/dL, 171 mg/dL, 172 mg/dL, 173 mg/dL, 174 mg/dL, 175 mg/dL, 176 mg/dL, 177 mg/dL, 178 mg/dL, 179 mg/dL, 180 mg/dL, 181 mg/dL, 182 mg/dL, 183 mg/dL, 184 mg/dL, 185 mg/dL, 186 mg/dL, 187 mg/dL, 188 mg/dL, 189 mg/dL, 190 mg/dL, 191 mg/dL, 192 mg/dL, 193 mg/dL, 194 mg/dL, 195 mg/dL, 196 mg/dL, 197 mg/dL, 198 mg/dL, 199 mg/dL, 200 mg/dL, 201 mg/dL, 202 mg/dL, 203 mg/dL, 204 mg/dL, 205 mg/dL, 206 mg/dL, 207 mg/dL, 208 mg/dL, 209 mg/dL, 210 mg/dL, 211 mg/dL, 212 mg/dL, 213 mg/dL, 214 mg/dL, 215 mg/dL, 216 mg/dL, 217 mg/dL, 218 mg/dL, 219 mg/dL, 220 mg/dL, 221 mg/dL, 222 mg/dL, 223 mg/dL, 224 mg/dL, 225 mg/dL, 226 mg/dL, 227 mg/dL, 228 mg/dL, 229 mg/dL, 230 mg/dL, 231 mg/dL, 232 mg/dL, 233 mg/dL, 234 mg/dL, 235 mg/dL, 236 mg/dL, 237 mg/dL, 238 mg/dL, 239 mg/dL, 240 mg/dL, 241 mg/dL, 242 mg/dL, 243 mg/dL, 244 mg/dL, 245 mg/dL, 246 mg/dL, 247 mg/dL, 248 mg/dL, 249 mg/dL, 250 mg/dL, 251 mg/dL, 252 mg/dL, 253 mg/dL, 254 mg/dL, 255 mg/dL, 256 mg/dL, 257 mg/dL, 258 mg/dL, 259 mg/dL, 260 mg/dL, 261 mg/dL, 262 mg/dL, 263 mg/dL, 264 mg/dL, 265 mg/dL, 266 mg/dL, 267 mg/dL, 268 mg/dL, 269 mg/dL, 270 mg/dL, 271 mg/dL, 272 mg/dL, 273 mg/dL, 274 mg/dL, 275 mg/dL, 276 mg/dL, 277 mg/dL, 278 mg/dL, 279 mg/dL, 280 mg/dL, 281 mg/dL, 282 mg/dL, 283 mg/dL, 284 mg/dL, 285 mg/dL, 286 mg/dL, 287 mg/dL, 288 mg/dL, 289 mg/dL, 290 mg/dL, 291 mg/dL, 292 mg/dL, 293 mg/dL, 294 mg/dL, 295 mg/dL, 296 mg/dL, 297 mg/dL, 298 mg/dL, 299 mg/dL, 300 mg/dL, 301 mg/dL, 302 mg/dL, 303 mg/dL, 304 mg/dL, 305 mg/dL, 306 mg/dL, 307 mg/dL, 308 mg/dL, 309 mg/dL, 310 mg/dL, 311 mg/dL, 312 mg/dL, 313 mg/dL, 314 mg/dL, 315 mg/dL, 316 mg/dL, 317 mg/dL, 318 mg/dL, 319 mg/dL, 320 mg/dL, 321 mg/dL, 322 mg/dL, 323 mg/dL, 324 mg/dL, 325 mg/dL, 326 mg/dL, 327 mg/dL, 328 mg/dL, 329 mg/dL, 330 mg/dL, 331 mg/dL, 332 mg/dL, 333 mg/dL, 334 mg/dL, 335 mg/dL, 336 mg/dL, 337 mg/dL, 338 mg/dL, 339 mg/dL, 340 mg/dL, 341 mg/dL, 342 mg/dL, 343 mg/dL, 344 mg/dL, 345 mg/dL, 346 mg/dL, 347 mg/dL, 348 mg/dL, 349 mg/dL, 350 mg/dL, 351 mg/dL, 352 mg/dL, 353 mg/dL, 354 mg/dL, 355 mg/dL, 356 mg/dL, 357 mg/dL, 358 mg/dL, 359 mg/dL, 360 mg/dL, 361 mg/dL, 362 mg/dL, 363 mg/dL, 364 mg/dL, 365 mg/dL, 366 mg/dL, 367 mg/dL, 368 mg/dL, 369 mg/dL, 370 mg/dL, 371 mg/dL, 372 mg/dL, 373 mg/dL, 374 mg/dL, 375 mg/dL, 376 mg/dL, 377 mg/dL, 378 mg/dL, 379 mg/dL, 380 mg/dL, 381 mg/dL, 382 mg/dL, 383 mg/dL, 384 mg/dL, 385 mg/dL, 386 mg/dL, 387 mg/dL, 388 mg/dL, 389 mg/dL, 390 mg/dL, 391 mg/dL, 392 mg/dL, 393 mg/dL, 394 mg/dL, 395 mg/dL, 396 mg/dL, 397 mg/dL, 398 mg/dL, 399 mg/dL, 400 mg/dL, 401 mg/dL, 402 mg/dL, 403 mg/dL, 404 mg/dL, 405 mg/dL, 406 mg/dL, 407 mg/dL, 408 mg/dL, 409 mg/dL, 410 mg/dL, 411 mg/dL, 412 mg/dL, 413 mg/dL, 414 mg/dL, 415 mg/dL, 416 mg/dL, 417 mg/dL, 418 mg/dL, 419 mg/dL, 420 mg/dL, 421 mg/dL, 422 mg/dL, 423 mg/dL, 424 mg/dL, 425 mg/dL, 426 mg/dL, 427 mg/dL, 428 mg/dL, 429 mg/dL, 430 mg/dL, 431 mg/dL, 432 mg/dL, 433 mg/dL, 434 mg/dL, 435 mg/dL, 436 mg/dL, 437 mg/dL, 438 mg/dL, 439 mg/dL, 440 mg/dL, 441 mg/dL, 442 mg/dL, 443 mg/dL, 444 mg/dL, 445 mg/dL, 446 mg/dL, 447 mg/dL, 448 mg/dL, 449 mg/dL, 450 mg/dL, 451 mg/dL, 452 mg/dL, 453 mg/dL, 454 mg/dL, 455 mg/dL, 456 mg/dL, 457 mg/dL, 458 mg/dL, 459 mg/dL, 460 mg/dL, 461 mg/dL, 462 mg/dL, 463 mg/dL, 464 mg/dL, 465 mg/dL, 466 mg/dL, 467 mg/dL, 468 mg/dL, 469 mg/dL, 470 mg/dL, 471 mg/dL, 472 mg/dL, 473 mg/dL, 474 mg/dL, 475 mg/dL, 476 mg/dL, 477 mg/dL, 478 mg/dL, 479 mg/dL, 480 mg/dL, 481 mg/dL, 482 mg/dL, 483 mg/dL, 484 mg/dL, 485 mg/dL, 486 mg/dL, 487 mg/dL, 488 mg/dL, 489 mg/dL, 490 mg/dL, 491 mg/dL, 492 mg/dL, 493 mg/dL, 494 mg/dL, 495 mg/dL, 496 mg/dL, 497 mg/dL, 498 mg/dL, 499 mg/dL, or 500 mg/dL, including all ranges and subranges therebetween.

The resulting correlational model can be used to convert known concentrations of one or more electrolyte into a blood glucose estimate. The correlational model is also sometimes referred to in the instant specification as a “glucose modification factor,” such that applying a glucose modification factor to a measured electrolyte concentration outputs a glucose concentration estimate.

### Reconciling Glucose Content Estimates

As previously mentioned, the apparatuses and methods described herein may estimate the glucose content of a blood sample using an optical measurement, e.g., light absorbance, of a single electrolyte (e.g., sodium, potassium or phosphate). In some embodiments, the present disclosure teaches apparatuses and methods of leveraging multiple electrolyte measurements to improve the accuracy and/or reliability of the glucose estimate to quantify the glucose content of the blood sample. Thus, in some embodiments, the glucose estimates are based on the measured concentration of two or more electrolytes, or three or more electrolytes. For example, in some embodiments, the glucose estimates are based on the measured concentration of sodium, potassium, and phosphate. In some embodiments, the glucose estimates are based on the measured concentration of sodium and potassium. In some embodiments, the present disclosure teaches reconciling the glucose content estimates based on separate electrolyte measurements, thereby quantifying the glucose level of in a blood sample.

In some embodiments the glucose content estimates from each of the electrolyte measurements undergo a data validation and reconciliation step. For example, in some embodiments, the concentration of each electrolyte is separately used to calculate a corresponding glucose estimate associated with that electrolyte, and all of the glucose estimates are averaged to provide a reported blood glucose level.

In other embodiments, the individual glucose estimates undergo different reconciliation steps, including process data reconciliation. For example, in some embodiments, the plurality of glucose estimates based on measurements of different electrolytes (e.g., sodium, potassium, and/or phosphate) can be weighted differently, or totally excluded from calculations depending on one or more factors. For example, in some embodiments, weight or inclusion of a glucose estimate from a particular electrolyte (from an absorbance measurement at a specific wavelength), will be based on the estimated reliability of that measurement. In some embodiments, weight or inclusion of a glucose estimate will be based on the presence/absence of redundant sensor data, the statistical analysis (e.g., distribution) of multiple measurements taken, and their relation to known biological ranges for each electrolyte. For example, measurements in which sequential samplings or redundant sensors reported conflicting values may be weighed lower than those with measurements deemed to be more reliable.

In some embodiments, weighing of each glucose estimate also considers the measured and historical relationship between the electrolytes, such that deviations from expected correlations in measurements can be flagged for reduced weight in the final reported glucose level (“quantified blood glucose level”). In some embodiments, the reconciling step comprises use of neural networks and/or machine learning.

### Apparatuses for Measuring Glucose Concentration

In some embodiments, the present disclosure describes an apparatus for measuring a blood glucose concentration. As used herein, the terms “apparatus” and “device” are used interchangeably throughout. The apparatus may include one or more processors configured to determine a concentration of one or more electrolytes (e.g., sodium, potassium, and/or phosphate), and estimate a blood glucose concentration based on the concentration of the one or more electrolytes. Specifically, the instant disclosure teaches an apparatus for non-invasively quantifying blood glucose, the apparatus comprising: a) a grip for releasably gripping a blood sample, the grip comprising first and second housings interconnected by a pivot configured to position the first and second housings relative to one another (e.g., by rotation) to releasably grip the blood sample inserted between the first and second housings, the first and second housings being in electrical communication with each other; b) a light emitting unit comprised within the first housing; the light emitting unit positioned so as to direct light towards the second housing, through the blood sample gripped between the first and second housings, the light emitting unit comprising at least two of: i) a light source capable of emitting light at 740-780 nm; ii) a light source capable of emitting light at 580-620 nm; iii) a light source capable of emitting light at 450-490 nm; and c) a light sensing unit comprised within the second housing, the light sensing unit comprising a photodetector configured to measure light transmittance from the light emitting unit, through the gripped blood sample.

In some embodiments, the apparatus of the present disclosure is designed as a clamp and includes two housing units connected by a hinge that releasably grip a body part or sample to measure a glucose concentration. In some embodiments, the clamp may resemble a pulse oximeter configured for attachment to a finger. The housings may be variously shaped to accommodate different types of body parts or samples. For example, in some embodiments, the inner facing portions of the first and second housing may be rounded so as to better accommodate a finger or blood samples in cylindrical containers. In other embodiments, the inner facing portions may have a flat surface to accommodate a flat-faced sample, or less-rounded body part (e.g., an ear). In some embodiments, the two housing units may include a hinge configured to couple the first housing to the second housing and pivot the first and second housings toward each other to close the clamp, and away from each other to open the clamp. The hinge may be any type of connector that is configured to move the first and second housings toward and away from each other. In some embodiments, the hinge is made from a rigid material, e.g., a metal, such as the hinge of a door. In other embodiments, the hinge is an integral hinge made from a polymer material. In yet further embodiments, the hinge comprises a soft material, e.g., a cloth or electrical cable.

In some embodiments, the hinge may be a spring-loaded hinge that biases the two housings toward each other to keep the clamp in a closed configuration. Instead of a spring, the clamping force between the two housings may come from an elastic or a non-elastic clip or clasp, or other structure configured to fix the position of the two housings at a predetermined location. In some embodiments, the clamping force between the two housings may be generated by magnets (e.g., via magnetic attraction).

Certain embodiments of the invention are designed to optically measure the concentration of one or more electrolytes in a blood sample or body part, and therefore include a light emitting unit positioned on one side of a sample, and a light sensing unit positioned on the opposite side of the sample. In these embodiments, the apparatus/device may include first and second housings positioned substantially parallel to each other, such that an inner surface of the first housing faces an inner surface of the second housing.

In some embodiments, the inner surface of the first housing includes the light emitting unit and second housing contains the light sensing unit. In some embodiments, the light emitting unit is positioned so as to direct light towards the light sensing unit in the inner surface of the other housing. The light sensing unit comprises at least one, at least two, or at least three light sources.

### Light Emitting Unit/Light Sources

In some embodiments, the instant disclosure teaches a light emitting unit including light sources for optically detecting potassium in blood. In some embodiments, the present disclosure teaches light sources configured to emit light at a wavelength between about 740-780 nm, including all values and sub-ranges therein. Accordingly, the instant disclosure teaches light sources configured to emit light having a wavelength of about 740 nm, 740 nm, 741 nm, 742 nm, 743 nm, 744 nm, 745 nm, 746 nm, 747 nm, 748 nm, 749 nm, 750 nm, 751 nm, 752 nm, 753 nm, 754 nm, 755 nm, 756 nm, 757 nm, 758 nm, 759 nm, 760 nm, 761 nm, 762 nm, 763 nm, 764 nm, 765 nm, 766 nm, 766.5 nm, 767 nm, 768 nm, 769 nm, 770 nm, 771 nm, 772 nm, 773 nm, 774 nm, 775 nm, 776 nm, 777 nm, 778 nm, 779 nm, or 780 nm, including all ranges and subranges therebetween. In some embodiments, the present disclosure teaches a light source configured to emit light at a wavelength of about 766.5 nm.

In some embodiments, the instant disclosure teaches light sources for optically detecting phosphate in blood. In some embodiments, the present disclosure teaches light sources configured to emit light at a wavelength between about 450-490 nm, including all values and sub-ranges therein. Accordingly, the instant disclosure teaches light sources configured to emit light having a wavelength of about 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, or 490 nm, including all ranges and subranges therebetween. In some embodiments, the present disclosure teaches a light source configured to emit light at a wavelength of about 470 nm.

In some embodiments, the instant disclosure teaches light sources for optically detecting sodium in blood. In some embodiments, the present disclosure teaches light sources configured to emit light at a wavelength between about 580-620 nm, including all values and sub-ranges therein. Accordingly, the instant disclosure teaches light sources configured to emit light having a wavelength of about 580 nm, 581 nm, 582 nm, 583 nm, 584 nm, 585 nm, 586 nm, 587 nm, 588 nm, 589 nm, 590 nm, 591 nm, 592 nm, 593 nm, 594 nm, 595 nm, 596 nm, 597 nm, 598 nm, 599 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, or 620 nm, including all ranges and subranges therebetween. Accordingly, the instant disclosure teaches light sources configured to emit light at a wavelength of about 595 nm.

In some embodiments, the instant disclosure teaches light sources configured to optically measure blood volume. In these embodiments, the light sources may be configured to emit light at a wavelength between about 620-680 nm, including all values and sub-ranges therein. For example, the light sources may be configured to emit light having a wavelength of about 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 651 nm, 652 nm, 653 nm, 654 nm, 655 nm, 656 nm, 657 nm, 658 nm, 659 nm, 660 nm, 661 nm, 662 nm, 663 nm, 664 nm, 665 nm, 666 nm, 667 nm, 668 nm, 669 nm, 670 nm, 671 nm, 672 nm, 673 nm, 674 nm, 675 nm, 676 nm, 677 nm, 678 nm, 679 nm, or 680 nm, including all ranges and subranges therebetween.

In some instances, it may be beneficial for the light sources to emit light at a wavelength of about 650 nm.

In some embodiments, the light source is a narrow-band light source. In some embodiments, the narrow-band light sources emit a spectra between 1 and 100 nm wide. That is, the narrow-band light source has at least 90% of its emissions within a 100 nm portion of the spectrum. In some embodiments, the narrow-band light source emits 90% of the light within a 50 nm portion of the spectra.

In some embodiments, the narrow-band light source is selected from the group consisting of: lasers, LEDs (Light Emitting Diodes), monochromators, and gas discharge lamps. In some embodiments, narrow-band emissions may be generated by using a broader spectrum light source coupled with filters. In some embodiments, the light source is an LED bulb.

### Light Sensing Unit

In some embodiments, the apparatus comprises a light sensing unit comprised within the second housing; the light sensing unit comprising a photodetector configured to measure light transmittance from the light emitting unit, through the gripped blood sample. Thus, in some embodiments the apparatus comprises one or more photodetectors (e.g., photodiode sensors).

In some embodiments the photodetector is configured to detect light transmitted through a blood sample. In some embodiments the photodetector is configured to detect light across a broad range of the electromagnetic spectrum. In some embodiments, the photodetector is configured to detect emissions across a narrower spectra. In some embodiments, the apparatus/device of the present disclosure comprises a separate photodetector for each light source. In other embodiments, a single photodetector may be configured to detect emissions from more than one light source (e.g., two, three, four light sources).

Any suitable type of photodetector may be employed in the apparatuses/devices described herein. In some embodiments, the photodetector may be a photodiode, a phototransistor, a photoresistor (LDR), a charge-coupled device (CCD), a complementary Metal-Oxide-Semiconductor (CMOS) sensor, a photomultiplier tube (PMT), an avalanche photodiode (APD), or a fiber optic sensor. In some embodiments, the photodetector is a photodiode.

As discussed above, certain embodiments of the apparatus contain redundant light sources and photodetectors. Therefore, in some embodiments, the apparatus comprises more than one light source configured to emit each type of light. For instance, in some embodiments, the apparatus may have multiple LED bulbs emitting light at about 766.5 nm. In some embodiments, those light sources are offset either in their X-Y location within the first housing, or positioned so as to emit light at a different angle. Similarly, the apparatus may contain multiple photodetectors (e.g., photodiodes) designed to detect light from multiple redundant LEDs. The different positioning of the multiple photodetectors and multiple redundant light sources (e.g., LEDs) can be helpful as a quality control, in case any one light source (LED bulb)/photodetector malfunctions. The different positioning can also be helpful, as it provides different light paths within the blood sample or tissue, providing duplicate measurements on absorbance, and reducing the chances that measurements for one wavelength are disrupted by contaminants or blocking tissue.

### Other Components

In some embodiments, the apparatuses of the present disclosure further comprises a power source. The power source may be a battery, a connection to an electrical power source, or a combination thereof. In some embodiments, the battery may be selected from the group of batteries consisting of: lithium-ion batteries, thin film lithium-ion batteries, lithium-ion polymer batteries, nickel-cadmium batteries, nickel metal hydride batteries, lead-acid batteries, and combinations thereof. The power source may comprise at least one rechargeable battery, and the at least one rechargeable battery may be operable to be recharged via at least one of the group consisting of: wireless charging, connection to an electrical power source, a motion-powered charging source, a pulse charging source, a solar charging source, a wind charging source, and combinations thereof.

In some embodiments, the apparatus of the present disclosure comprises a display. In some embodiments, the display can be configured as a plasma, liquid crystal display (LCD), light emitting diode (LED), field emission display (FED), surface-conduction electron-emitter display (SED), organic light emitting diode (OLED), or flexible OLED display device. The display may be configured, manufactured, produced, or assembled based on the descriptions provided in U.S. Patent Publication Nos. 2007-247422, 2007-139391, 2007-085838, 2006-096392, or 2010-295812 or U.S. Pat. No. 7,050,835 all herein incorporated by reference as if fully set forth. In the case of a flexible or bendable display device, the electronic display may be configured and assembled using organic light emitting diodes (OLEDs), liquid crystal displays using flexible substrate technology, flexible transistors, field emission displays (FED) using flexible substrate technology, or the like. In some embodiments, the display can also be configured as a three-dimensional (3D), electronic paper (e-paper), or electronic ink (e-ink) display.

In some embodiments, the display can be configured as a touch, multi-touch, multiple touch, or swipe screen display using resistive, capacitive, surface-acoustic wave (SAW) capacitive, infrared, strain gauge, optical imaging, dispersive signal technology, acoustic pulse recognition, frustrated total internal reflection, magneto-strictive technology, or the like.

In some embodiments, the apparatus further comprises an electronic storage device. In some embodiments, the storage device can be any disk based or solid-state memory device for storing data.

In some embodiments, the apparatus comprises a microcontroller board. In some embodiments, any commercially available type of microcontroller board may be used including, but not limited to an 8051, peripheral interface controllers (PICx) and, a Alf-Egil Bogen and Vegard Wollan's RISC (AVR) processor. Microcontroller boards that may be employed include, but are not limited to, an Arduino Uno, a ESP-32, a Raspberry Pi, an Intel Edison, a Udoo Neo, a LightBlue Bean, an Adafruit Flora, a Tessel, a Particle Photon and a Mediatek Linkit One.

### Apparatus Operation

In some embodiments, the microcontroller comprises instructions for estimating blood glucose contents. In some embodiments, the microcontroller-executable instructions may be in a machine language compatible with the microcontroller board being used including, but not limited to C, C++, Python and JavaScript. In one aspect, the microcontroller board is a ESP-32 and the microcontroller-executable instructions are in C++. The library of algorithms may be written and stored in the non-volatile memory using commercial or open-resource programming code editors including, but not limited to Arduino Integrated Development Environment, Atom, Notepad++, Visual Studio Code and Komodo Edit. One of skill in this art would be well aware of using these programming code editors and select an editor that is compatible with the microcontroller board. In one aspect, the microcontroller board is an ESP-32, the microcontroller-executable instructions are in C++ and the programming code editor is an Arduino Integrated Development Environment (IDE) editor.

Illustrative pseudocode demonstrating possible instructions that could be programmed into the microcontroller are provided below. This pseudocode is provided for illustrative purposes only, and is not intended to be construed as limiting to the instant invention.
1. Define OLED display dimensions (SCREEN_WIDTH, SCREEN_HEIGHT)
2. Define OLED reset pin (OLED_RESET)
3. Initialize display (with screen dimensions and reset pin)
4. Declare sensor and LED pins (sensorPin, potassiumLed, phosphateLed)
5. Set baseline intensities for potassium and phosphate LEDs (baselineIntensityPotassium, baselineIntensityPhosphate)
setup:
   a. Initialize serial communication
   b. Set sensorPin as input
   c. Set potassiumLed and phosphateLed as output
   d. Initialize display
      i. If display initialization fails, print error and halt execution loop:
a. For potassium measurement:
   i. Turn on potassium LED
   ii. Wait for 1 second (optional stabilization delay)
   iii. Read sensor value for potassium
   iv. Turn off potassium LED
   v. Calculate absorbance for potassium using the formula: −log 10(sensor reading/baseline intensity)
b. Wait for 1 second (delay between measurements)
c. For phosphate measurement:
   i. Turn on phosphate LED
   ii. Wait for 1 second (stabilization delay)
   iii. Read sensor value for phosphate
   iv. Turn off phosphate LED
   v. Calculate absorbance for phosphate using the formula: −log 10(sensor reading/baseline intensity)
d. Solve for concentration (x) for potassium and phosphate using given quadratic equation
e. Reconcile (e.g., calculate the average of x values for potassium and phosphate)
f. Display and print the average x value
g. Wait for 2 seconds before next measurement cycle calculate Absorbance: (separate function)
   Input: intensity, baselineIntensity
   Calculate transmittance: intensity/baselineIntensity
   Calculate absorbance: −log 10(transmittance)
   Return absorbance solveQuadratic: (separate function)
   Input: coefficients a, b, c (for quadratic equation ax\hat{ }2+bx+c=0)
   Calculate the discriminant: sqrt(b\hat{ }2-4ac)
   If discriminant >0, indicate an error (specific to the original code's context)
   Calculate and return the first root: (−b+sqrt(discriminant))/(2a)

### Fluid Samples

As previously mentioned, the methods and apparatuses may determine (e.g., by estimating, as described herein) glucose concentrations in various types of fluid samples such as blood, plasma, urine, cerebrospinal fluid, pleural fluid, ascitic fluid, or sweat. In some embodiments, the instant disclosure teaches methods and apparatuses for estimating the glucose content of blood samples. In some embodiments, the blood sample can be circulating blood within an animal. Thus, in some embodiments, the blood sample can refer to a body part in which the optical measurements are made. In some embodiments, the body part is selected from the group consisting of a finger, a toe, an ear, a lip, a nose, an eye lid, tongue, gums, and a skin fold. The methods and apparatuses of the present disclosure may analyze ex-vivo blood samples. Accordingly, in some embodiments, the blood sample is a container of blood previously harvested from an animal. In some embodiments, the sample is in a clear container that does not affect transmission of light at the wavelength used to measure each of the electrolytes.

### Systems for Measuring Glucose Concentration

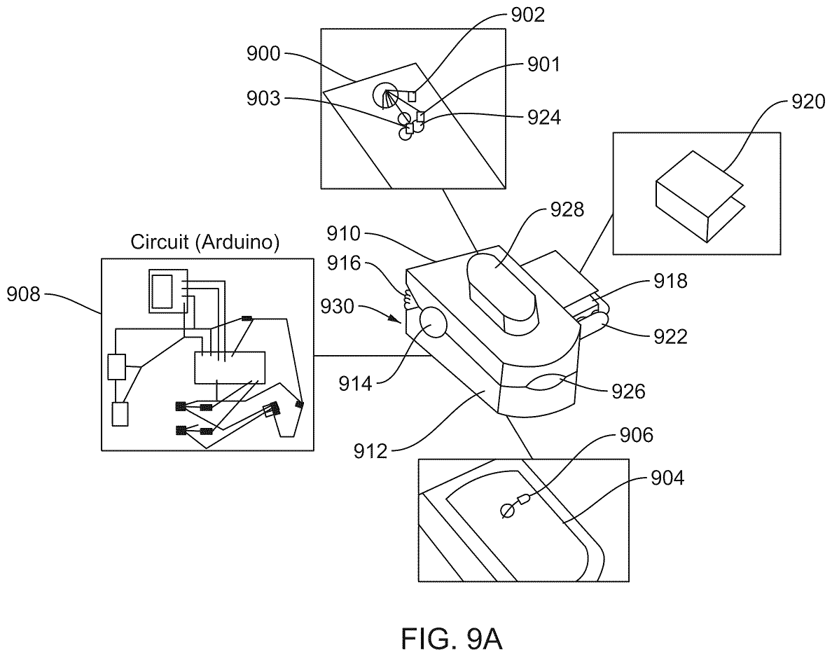

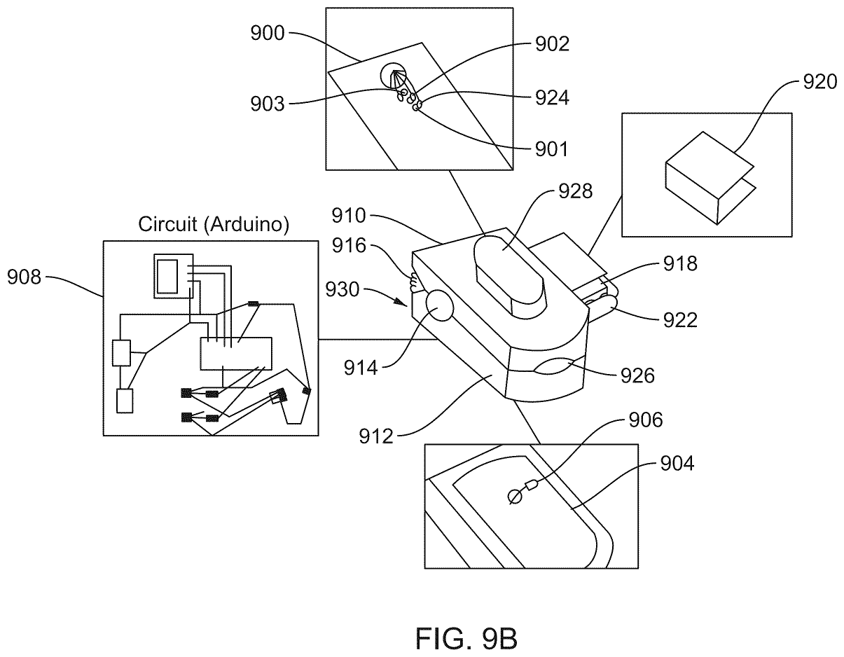

Also described herein are systems for measuring glucose concentrations. The systems may be configured to be portable and non-invasive, such that they may be used at home as well as in a clinic or hospital setting. In some embodiments, as shown in FIGS. 9A and 9B, which are further described below in Example 3, the system may include a clamp configured to attach to a body site or container holding a fluid sample (e.g., vial, tube), where the clamp comprises a first housing coupled to a second housing. The first and second housings may be configured as previously described herein. For example, the first housing may include a light emitting unit comprising at least two light sources, and the second housing may include one or more photodetectors. The systems may also include one or more processors configured to receive data from the one or more photodetectors indicative of a concentration of one or more electrolytes in a fluid at the body site and determine a glucose concentration of the fluid based on the concentration of the one or more electrolytes. Exemplary electrolytes include without limitation, sodium, potassium, and phosphate. The one or more processors may be contained in the first housing and/or the second housing, or may be located outside the housings and coupled to the one or more photodetectors via a wired or wireless connection (e.g., WiFi, Bluetooth).

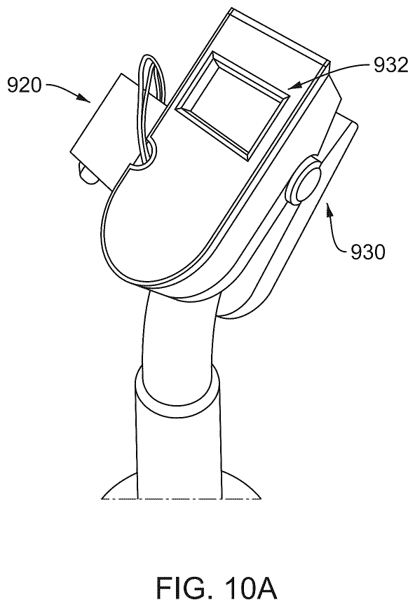

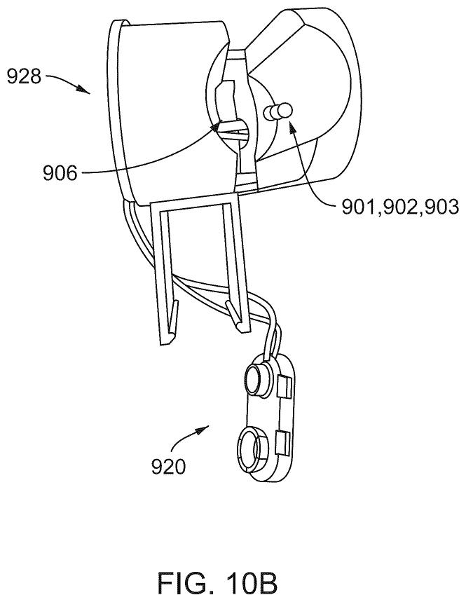

The first and second housings of any of the embodiments described herein may have various dimensions. In some embodiments, the first housing may have a length between about 2.0 cm to about 10 cm, including all values and sub-ranges therein. For example, the first housing length may be about 2.0 cm, about 2.5 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, about 8.0 cm, about 8.5 cm, about 9.0 cm, about 9.5 cm, or about 10 cm. In some embodiments, a width of the first housing may be between about 2.0 cm to about 6.0 cm, including all values and sub-ranges therein. For example, the first housing width may be about 2.0 cm, about 2.5 cm, about 3.0 cm, about 3.5 cm, about 4.0 cm, about 4.5 cm, about 5.0 cm, about 5.5 cm, or about 6.0 cm. The second housing may have the same length and width as the first housing. Alternatively, a length and/or width of the second housing may be different from the first housing. The first and second housings may also be variously shaped. For example, the housings may be rectangular, square, circular, ovular, or triangular. In some instances, as shown in FIGS. 9A, 9B, and 10A, which are further described below in Example 3, one end of each housing may have a straight edge, and the opposite end of each housing may have a curved edge.

The first and second housings of any of the embodiments described herein may be formed from a polymeric material and/or a metal material. In some embodiments, the polymeric material may be ABS (acrylonitrile butadiene styrene), polycarbonate, polyethylene, polypropylene, or combinations thereof. When a metal or metal alloy is used, the material may be stainless steel, aluminum, nickel, or combinations thereof. The housings may be made by such process as injection molding, casting, 3D printing, or CNC (computer numerical control) machining.

Coupling of the first housing to the second housing may be accomplished via any suitable connector. In some embodiments, the connector comprises a hinge. The hinge may be a butt hinge, pivot hinge, or an integral hinge. In some embodiments, the hinge comprises one or more springs (e.g., a spring-loaded hinge). The hinge may generally be configured to transition the clamp from a closed configuration to an open configuration. Put another way, the hinge may be configured to move the first and second housings toward each other to close the clamp, and move the first and second housings away from each other to open the clamp.

In some embodiments, a hinge may be disposed at one end of the housings. In other embodiments, a hinge may be disposed at both ends of the housings, e.g., at the proximal and distal ends (thus, two hinges are employed in the clamp). In some embodiments, a hinge included at a proximal end of each of the first and second housings forms a closed proximal clamp end. When the hinge is a pivot hinge, rotation of the first and second housings about a pivot point of the hinge separates the distal ends of the housings to thus create an open distal clamp end. In some embodiments, the hinges may bias the clamp to its closed configuration so that the first and second housings may securely attach to the target body site (e.g., finger, ear, lip) or a container configured to hold blood or other fluid sample. In the open configuration, the clamp in these embodiments may accept the container or tissue from the body site such that the tissue is positioned between the first and second housings.

Instead of a hinge, in some embodiments, the first and second housings are attached to each other by an adhesive. In other embodiments, the first and second housing are attached to each other by a snap-fit connection. In yet further embodiments, the first and second housings are attached using mechanical fasteners (e.g., screws, nuts, bolts), magnets, welding, or soldering.

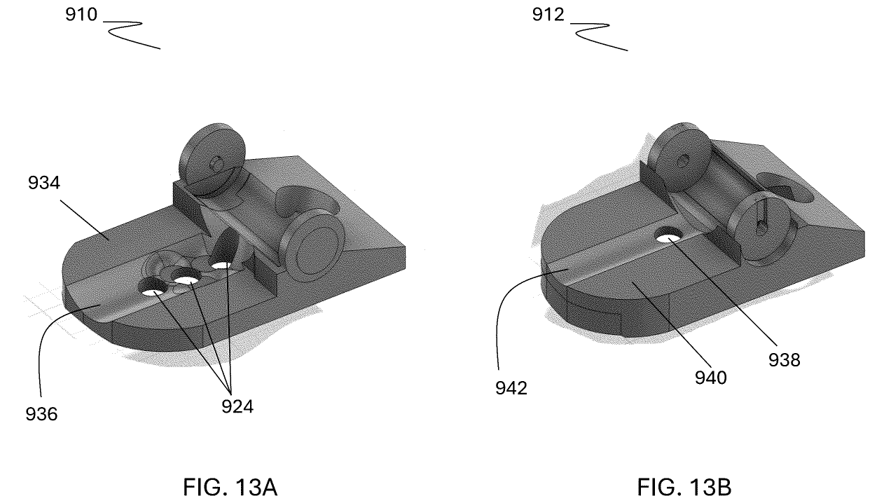

In some embodiments, the inner surface of one or more of the first and second housings may include a cut-out, groove, recess (e.g., as further described below for FIGS. 13A and 13B in Example 3), or channel configured to help hold a body part (or tissue of the body part) between the first and second housings when the clamp is in a closed configuration. The recess may be an elongate recess. The length and width of the recess may vary depending on the body part or tissue of interest to be held between the first and second housings. In some embodiments, the inner surface of one or more of the first and second housings includes a compressible and/or conformable material to provide a cushion between the body site and the housings. The compressible and/or conformable material may be optically clear so as not to interfere with obtaining optical measurements, and layered partially or entirely over the inner surface. In some embodiments, the compressible and/or conformable material comprises silicone.

The first and second housings may be configured as cases that entirely or partially enclose various system components. In some embodiments, the first and second housings may be configured as first and second arms including one or more cut-outs or openings into which light sources and/or photodetectors may be at least partially placed, as shown in FIGS. 13A and 13B, which are further described below in Example 3. For example, in some embodiments, the first housing may contain a light emitting unit that includes a plurality of lights sources (e.g., two, three, four, five light sources). The light sources may be light-emitting diodes (LEDs). In some embodiments, the light emitting unit includes at least two light sources, each comprising a light-emitting diode (LED). In other embodiments, the light emitting unit includes at least three light sources, each comprising a light-emitting diode (LED). The first housing may include one or more openings into which the light sources are placed. The number, size, shape, and location of the openings may generally correspond to the number, size, shape, and location of the light sources employed. In some embodiments, the first housing includes two openings. In other embodiments, the first housing includes three openings. In some embodiments, the openings in the first housing are circular in shape. Similarly, the second housing may include one or more openings into which the photodetectors are placed. The number, size, shape, and location of these opening may generally correspond to the number, size, shape, and location of the photodetectors that are used. In some embodiments, the second housing includes one opening. In other embodiments, the second housing includes two openings. In some embodiments, the openings in the second housing are circular in shape.

The light emitting unit of the systems may be configured as previously described herein and emit light at a wavelength between about 740 nm to about 780 nm, between about 580 nm to about 620 nm, between about 450 nm to about 490 nm, and/or between about 625 nm to about 675 nm. The light that is emitted may have a wavelength or wavelength range predetermined by, e.g., a controller of the system. For example, light emitted at a predetermined wavelength between about 740 nm to about 780 nm may be used to detect potassium in a blood sample. Light emitted at a predetermined wavelength between about 580 nm to about 620 nm may be used to detect sodium in a blood sample. Light emitted at a predetermined wavelength between about 450 nm to about 490 nm may be used to detect phosphate in a blood sample. Although blood samples are mentioned here, it is understood that the systems may analyze any fluid sample containing potassium, sodium, and/or phosphate with their associated wavelengths of light. For example, fluid samples such as plasma, urine, cerebrospinal fluid, pleural fluid, ascitic fluid, or sweat may be analyzed.

In some embodiments, the second housing includes one or more photodetectors. For example, as previously mentioned, the one or more photodetectors may be a phototransistor, a photoresistor (LDRs), a charge-coupled device (CCD), a complementary Metal-Oxide-Semiconductor (CMOS) sensor, a photomultiplier tube (PMT), an avalanche photodiode (APD), or a fiber optic sensor. In some embodiments, the one or more photodetectors may comprise a photodiode. In use, light emitted by the one or more light sources in the light emitting unit travels through the fluid sample including potassium, sodium, and/or phosphate, e.g., a blood sample, and is then detected by the one or more photodetectors. The one or more photodetectors may convert the detected light into data (e.g., an electrical signal), which is transmitted to one or more processors for further analysis. For example, in some embodiments, the one or more processors is configured to apply a glucose modification factor, obtained as described elsewhere herein, to the received data from the one or more photodetectors that is indicative of the concentration of the one or more electrolytes in order to determine the glucose concentration of the fluid sample. The one or more processors may also be configured to apply correlation models and/or perform data validation or reconciliation, as previously described herein. For example, the one or more processors may be configured to execute instructions from a controller, such as a microcontroller board described elsewhere herein.

Additionally or alternatively, the one or more processors may be configured to apply a machine learning algorithm to the received data from the one or more photodetectors indicative of the concentration of one or more electrolytes in order to determine the glucose concentration of the fluid sample. The machine learning algorithm may comprise a random forest, a boosted decision tree, a classification tree, a regression tree, a bagging tree, a neural network, a rotation forest, or combinations thereof. When a neural network is used, the neural network may comprise a convolutional neural network (CNN), a deep convolutional neural network, a recurrent neural network (RNN), or combinations thereof.

In some embodiments, at least a portion of the clamp is disposable. In other embodiments, the entire clamp is disposable. The clamp may be configured to releasably attach to various body areas/sites including without limitation, a finger, toe, ear, lip, tongue, arm, wrist, and ankle, and determine a glucose concentration of blood based on the electrolyte concentrations of blood at those areas/sites.

In some embodiments, the system further includes a display. The display may visually present various types of information, e.g., time, date, wavelength of light being used, electrolyte concentration, and/or the determined glucose concentration. The display may provide a visual alert (e.g., a flashing and/or colored light) and/or an audible alert if the determined glucose concentration is above a predetermined level. In some embodiments, the display may include a user interface such as a touch screen or buttons, which may be used to power on/off the system, select light emitters, edit information, or select the information to be presented on the display.

The systems described herein may further include a power source. The power source may be a rechargeable or non-rechargeable battery. In some embodiments, the power source is removably secured to the first housing or the second housing, e.g., using a battery clip.

Methods for non-invasively measuring glucose concentrations are also described herein. In some embodiments, the methods may include attaching a clamp to a body site, where the clamp comprises at least two light sources and one or more photodetectors, and irradiating the body site with light wavelengths associated with two or more electrolytes. The electrolytes may be sodium, potassium, and/or phosphate. Furthermore, the method may include receiving data from the one or more photodetectors indicative of a concentration of one or more electrolytes in a fluid at the body site, and determining a glucose concentration of the fluid based on the concentration of the one or more electrolytes. Although a clamp is mentioned here, it is understood that the methods may be performed by any one of the apparatuses or devices (e.g., clamps, grips) described herein. Additionally, although the clamp is described as being attached to a body site, in some embodiments, the clamp (or other apparatus/device) may be attached to a container configured to hold a fluid sample (e.g., a vial, tube).

In some embodiments, the methods include irradiating the fluid sample with light at wavelengths associated with sodium and potassium. In other embodiments, the methods include irradiating the fluid sample with light at wavelengths associated with sodium and phosphate. In further embodiments, the methods include irradiating the fluid sample with light at wavelengths associated with potassium and phosphate. The light wavelengths associated with sodium may be between about 580 nm to about 620 nm. The light wavelengths associated with potassium may be between about 740 nm to about 780 nm. The light wavelengths associated with phosphate may be between about 450 nm to about 490 nm. In some embodiments, the method includes illuminating a body site (e.g., a body part, body tissue) with light at one or more predetermined wavelengths, transmitting the light through a fluid sample at the body site, and detecting the transmitted light via a photodetector. Output signals from the photodetector may include data (e.g., related to one or more electrolytes) representative of the glucose concentration of the fluid sample. The output signals may be transmitted to one or more processors for further analysis to determine a glucose concentration as further described below and elsewhere herein.

The methods may include using one or processors to receive the data from the one or more photodetectors indicative of a concentration of the one or more electrolytes in the fluid at the body site. After receipt of the data, in some embodiments, the one or more processors are configured to apply a glucose modification factor and/or correlation models to the data, and/or perform data validation or reconciliation, as previously described herein.

Additionally or alternatively, the methods may employ one or more processors configured to apply a machine learning algorithm to the received data indicative of the concentration of one or more electrolytes in order to determine the glucose concentration of the fluid sample. The machine learning algorithm may comprise a random forest, a boosted decision tree, a classification tree, a regression tree, a bagging tree, a neural network, a rotation forest, or combinations thereof. When a neural network is used, the neural network may comprise a convolutional neural network (CNN), a deep convolutional neural network, a recurrent neural network (RNN), or combinations thereof.

The methods may be performed on any fluid having sodium, potassium, or phosphate, at any body site, or on any container in which the apparatuses/devices described herein can be attached. When attachment is to a body site, the body site may be a finger, toe, ear, lip, tongue, arm, wrist, or ankle. Exemplary fluids for analysis may include without limitation, blood, plasma, urine, cerebrospinal fluid, pleural fluid, ascitic fluid, and sweat. In some embodiments, a glucose concentration is determined for a sample of blood. As previously mentioned, this glucose concentration is based on concentrations of sodium, potassium, and/or phosphate in the blood sample.

The methods may be used to non-invasively monitor blood glucose levels in patients diagnosed with diabetes (including type 1 and type 2 diabetes), gestational diabetes, or pre-diabetes. In some embodiments, the methods may be used to obtain blood glucose levels during routine examinations. The non-invasive nature of the methods (and systems) described herein avoids finger-sticks and thus may improve patient compliance with blood glucose monitoring. In turn, improved patient compliance may help patients adjust their diet, exercise, and/or medication dosing in a quick and efficient manner to avoid spikes in blood glucose levels.

### EXAMPLES

The following examples are illustrative only and should not be construed as limiting the disclosure in any way.

### Example 1: Determining Absorbance of Electrolytes in Aqueous Solution

The absorbance of electrolytes in aqueous solutions was analyzed by spectroscopy to determine useful detection wavelengths. Potassium, sodium, and phosphate electrolytes were irradiated with various light wavelengths, and standard curves were created from their respective absorption values to evaluate the relationship between absorbance and electrolyte concentration.

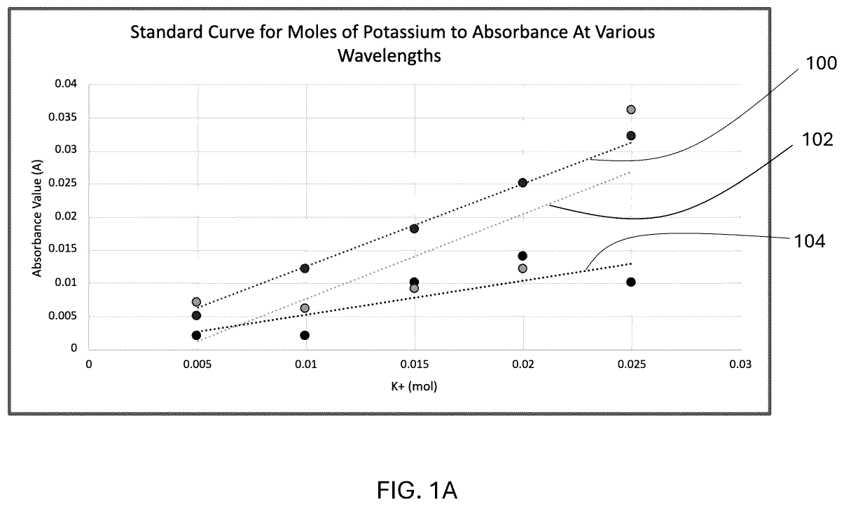

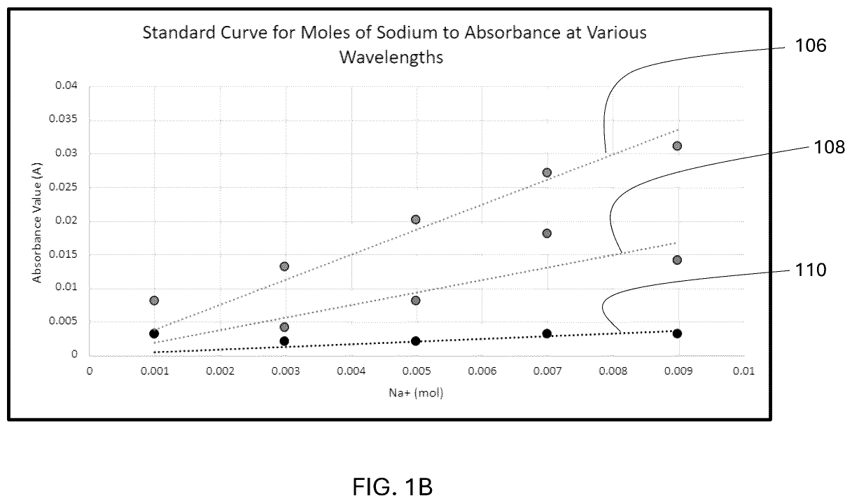

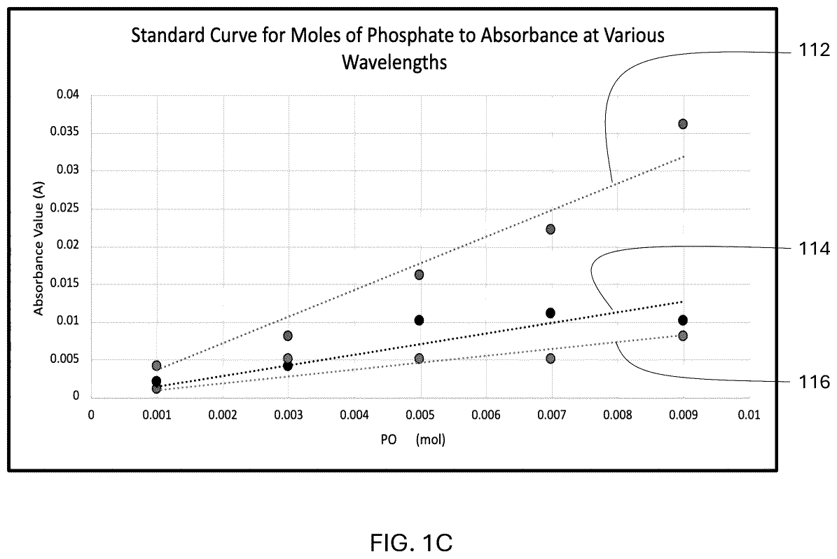

FIGS. 1A-1C show the absorbance of potassium (FIG. 1A), sodium (FIG. 1B), and phosphate (FIG. 1C) across a range of concentrations at three different wavelengths. For potassium, the three different wavelengths were 760.0 nm, 766.5 nm, and 770.0 nm. For sodium, the three different wavelengths were 595.0 nm, 600.0 nm, and 605.0 nm. For phosphate, the three different wavelengths were 460.0 nm, 465.0 nm, and 470.0 nm. Standard curves were generated for each set of test absorbance, and the coefficient of determination (R²) was determined for each set of data points, as shown in Table 2.

TABLE 2
Standard curve and coefficient determination for
electrolyte absorbance at various wavelengths

| | Potassium | | Sodium | | Phosphate |
|---|---|---|---|---|---|
| 760.0 nm | y = 0.9964x; | 595.0 nm | y = 3.727x; | 460.0 nm | y = 9152x; |
| | R² = 0.8499 | | R² = 0.9868 | | R² = 0.8915 |
| 766.5 nm | y = 1.247x; | 600.0 nm | y = 1.861x; | 465.0 nm | y = 1.400x; |
| | R² = 0.9986 | | R² = 0.9079 | | R² = 0.9484 |
| 770.0 nm | y = 0.5164x; | 605.0 nm | y = 0.4061x; | 470.0 nm | y = 3.533x; |
| | R² = 0.9075 | | R² = 0.7773 | | R² = 0.9905 |

Wavelengths corresponding to the R² value indicating best fit were selected to detect the presence of exemplary electrolytes in future experimentation. Based on the standard curves generated by irradiation at 766.5 nm (100), 760.0 nm (102), and 770.0 nm (104), as shown in FIG. 1A, and their corresponding R² values provided in Table 2, the wavelength having the best fit for potassium was 766.5 nm (R² value of 0.9986). Based on the standard curves generated by irradiation at 595.0 nm (106), 600.0 nm (108), and 605.0 nm (110), as shown in FIG. 1B, and their corresponding R² values provided in Table 2, the wavelength having the best fit for sodium was 595.0 nm (R² value of 0.9868). Based on the standard curves generated by irradiation at 460.0 nm (112), 465.0 nm (114), and 470.0 nm (116), as shown in FIG. 1C, and their corresponding R² values provided in Table 2, the wavelength having the best fit for phosphate was 470 nm (R² value of 0.9905).

### Example 2: Detecting Glucose in Silkworm Hemolymph

Hemolymph, the fluid that circulates throughout the body of the silkworm, can provide a rapid and dose-dependent response to dietary glucose. Accordingly, in this example, silkworms were selected as the model organism for determination of glucose levels.

Growth conditions: Silkworm eggs were incubated at 28-29° C. After hatching, temperature was reduced to 27° C. Food was replaced every 12 hours during the first three malting phases and reduced to every 24 hours during the last 2 hours. Worms were either transferred to a new petri dish every 120 hours or their defecation was completely removed every 72 hours. Experimentation commenced after the first molting phase was complete.

Induction of Hyperglycemia: Silkworm hemolymph was evaluated for its capacity to provide a standard for non-invasive glucose monitoring. To examine the feasibility of using silkworm hemolymph as a glucose test standard, hyperglycemia was induced by adding increasing amounts of sugar to silkworm diets. The effects of a sugar diet on growth and mortality were compared to a control group (no sugar meals) to determine the optimal level of sugar for a hemolymph standard containing glucose.

Test diets: Silkworms were allocated to either a control group (no sugar meal) or a sugar-fed group in which silkworms were fed a sugar-to-food ratio of 1:12, or 1:9. Each group included approximately 20 worms each. Table 3 presents the physical appearance and behavior of silkworms in each group.

TABLE 3
Silkworm experimental groups

| Diet | Observation |
|---|---|
| No sugar diet (negative control) | Minimal deaths, healthy, moving |
| 1:12 sugar-to-food ratio | Slightly more fatigued, larger, |
| | minimal deaths |
| 1:9 sugar-to-food ratio | Fatigued, significant deaths, |
| | more food eaten |

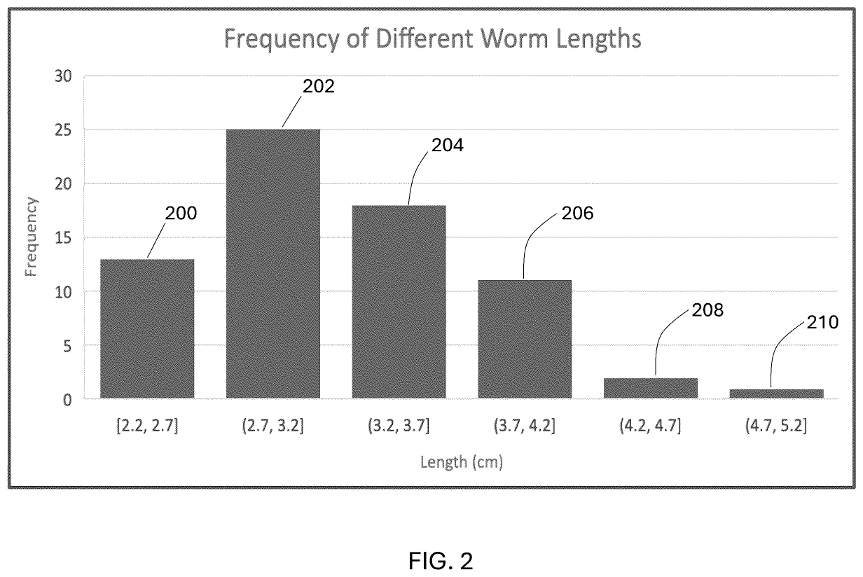

As shown in FIG. 2, worms in the different groups grew to various lengths, adding diversity to the sample size and strengthening correlative data. The lengths were divided into ranges of 2.2 cm to 2.7 cm (200), 2.7 cm to 3.2 cm (202), 3.2 cm to 3.7 cm (204), 3.7 cm to 4.2 cm (206), 4.2 cm to 4.7 cm (208), and 4.7 cm to 5.2 cm (210).

Hemolymph Isolation: To remove hemolymph, the worms were frozen for 5 minutes to render them unconscious. The worms were then sliced in half and placed in separate centrifuge tubes. 200 μL of distilled water was added to each tube. The mixture was centrifuged for 15 minutes at 12000 rpm. The resulting supernatant was placed in a separate centrifuge tube and re-centrifuged at 12000 rpm for 15 minutes to remove excess tissue. The supernatant was then extracted and diluted with 3 mL of distilled water to produce a 4 mL “diluted solution.” The absorbances of each of the silkworm trials were recorded at the wavelengths identified in Example 1. As a control, approximately 5 μL of the diluted solution was applied to a glucometer test strip. Finally, the glucose concentration displayed on the glucometer was recorded.

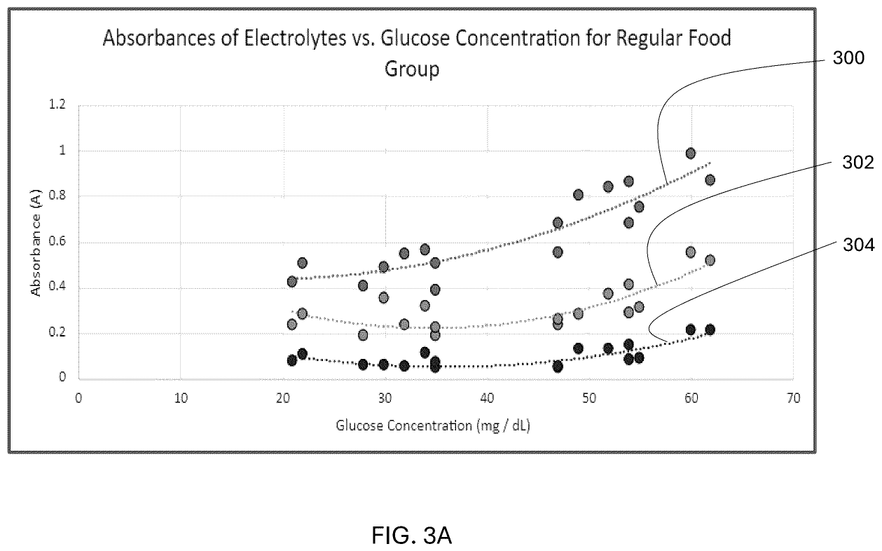

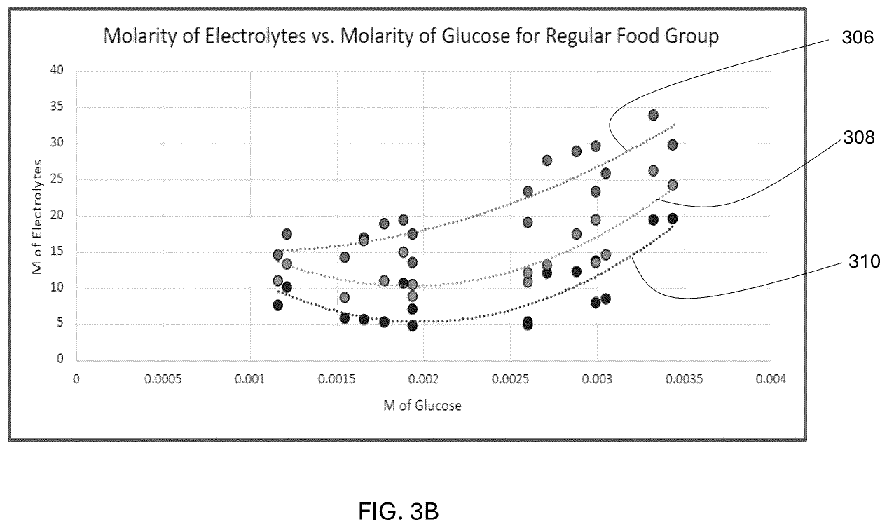

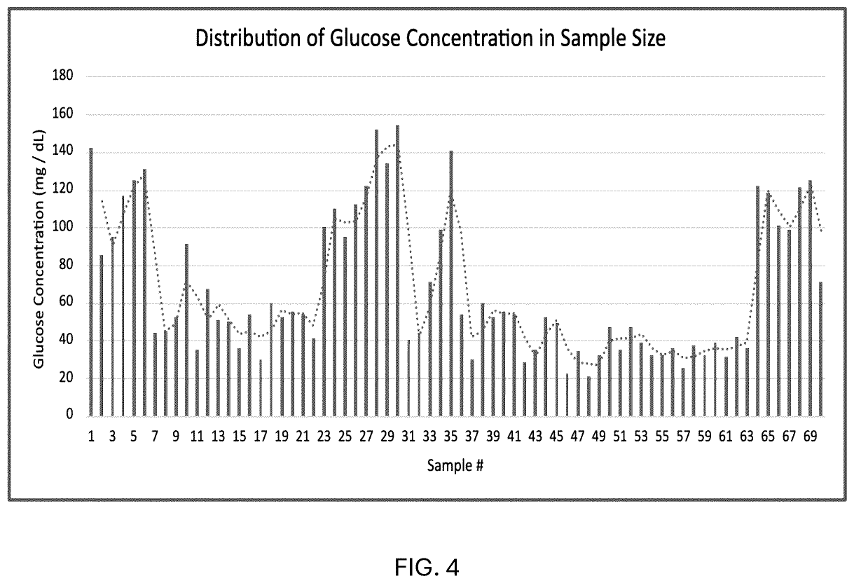

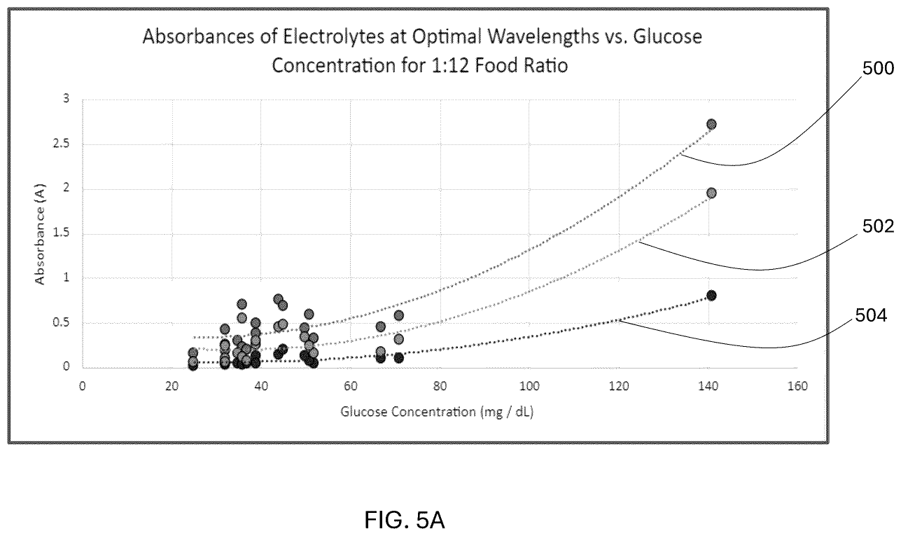

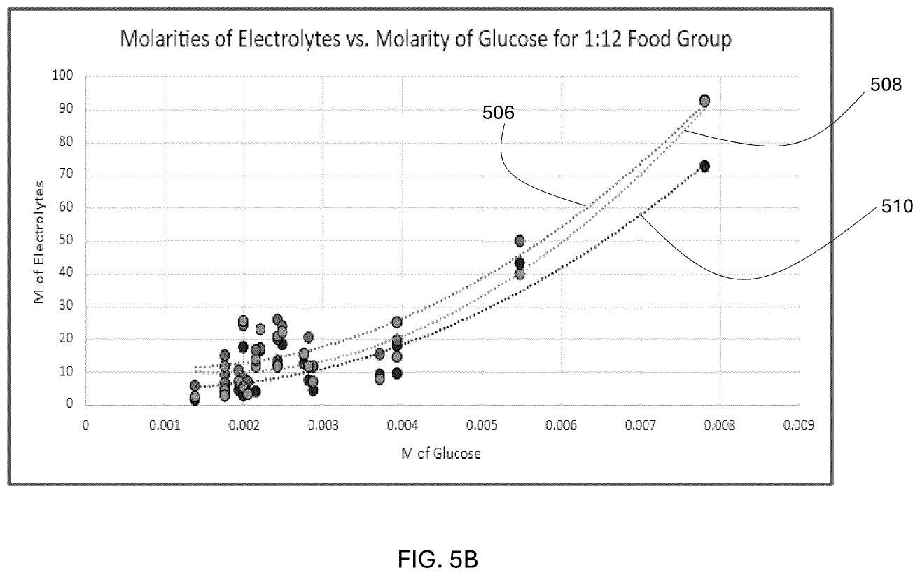

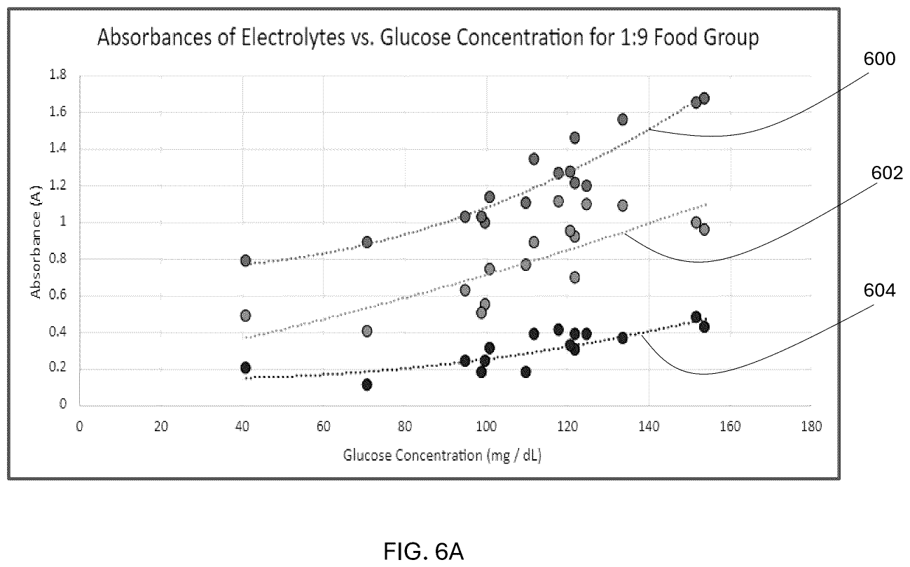

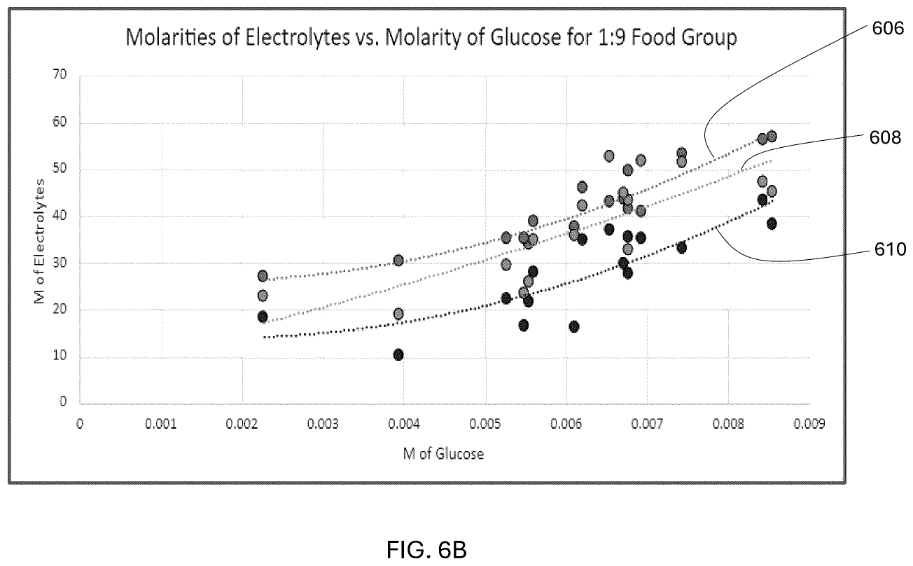

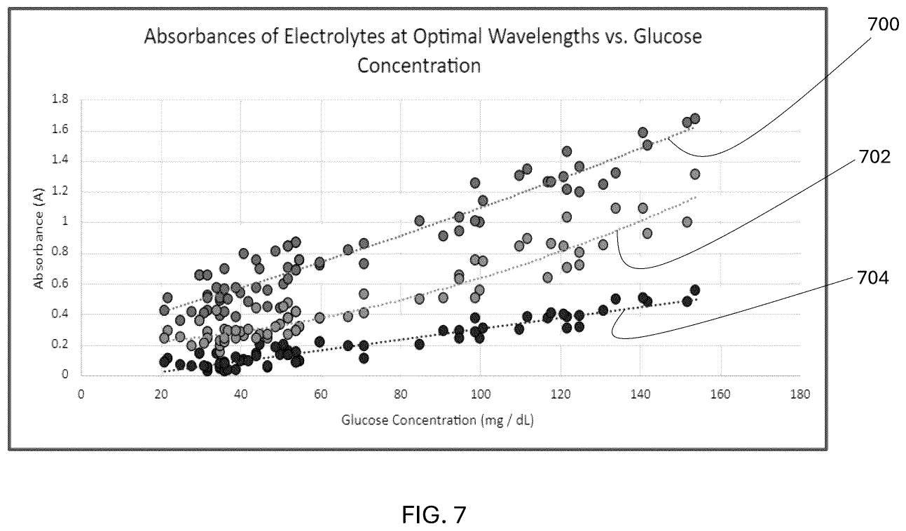

FIG. 4 shows the distribution of glucose concentrations across tested control samples from silkworms that were fed a non-sugar diet, which ranged from approximately 25 mg/dL to 155 mg/dL. These samples with known glucose concentrations measured via a glucometer were irradiated with the best fit wavelengths for phosphate, sodium, and potassium (as determined in Example 1), their absorbances plotted, and regression curves generated for determination of a glucose modification factor (curve 300 for phosphate, curve 302 for sodium, and curve 304 for potassium), as shown in FIG. 3A. As shown in FIG. 3B, the known concentrations were plotted against their corresponding optically-measured electrolyte concentrations, and regression curves generated (curve 306 for phosphate, curve 308 for sodium, and curve 310 for potassium). In this manner, regression models (correlation models) were built for each series of measurements, and saved for future application to test reads. These models were developed to test samples from silkworms in different sugar-fed groups, to cover all glucose ranges. For example, in FIG. 5A, samples from silkworms fed a 1:12 sugar-to-food ratio diet were irradiated with the best fit wavelengths for phosphate, sodium, and potassium (as determined in Example 1), their absorbances plotted, and regression curves generated for determination of a glucose modification factor (curve 500 for phosphate, curve 502 for sodium, and curve 504 for potassium). As shown in FIG. 5B, the known glucose concentrations were plotted against their corresponding optically-measured electrolyte concentrations, and regression curves generated (curve 506 for phosphate, curve 508 for sodium, and curve 510 for potassium). Referring to FIG. 6A, samples from silkworms fed a 1:9 sugar-to-food ratio diet were irradiated with the best fit wavelengths for phosphate, sodium, and potassium (as determined in Example 1), their absorbances plotted, and regression curves generated for determination of a glucose modification factor (curve 600 for phosphate, curve 602 for sodium, and curve 604 for potassium). As shown in FIG. 6B, the known glucose concentrations were plotted against their corresponding optically-measured electrolyte concentrations, and regression curves generated (curve 606 for phosphate, curve 608 for sodium, and curve 610 for potassium). FIG. 7 is a graph that shows the combined absorbances from the 1:12 and 1:9 sugar-to food ratio silkworm groups based on irradiation with best fit wavelengths for phosphate, sodium, and potassium (as determined in Example 1), and regression curves based thereon (curve 700 for phosphate, curve 702 for sodium, and curve 704 for potassium).

### Example 3: Electrolyte Detector with Glucometer Output

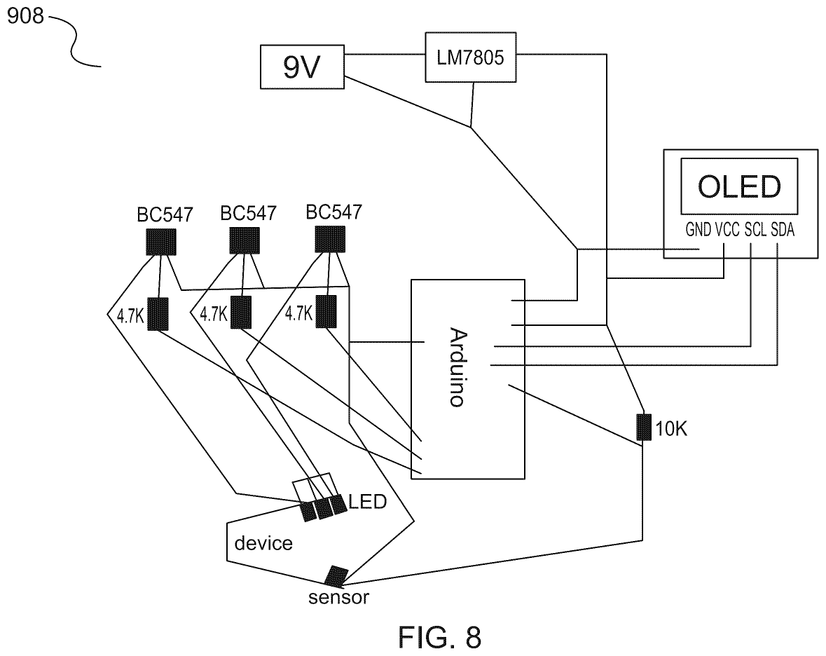

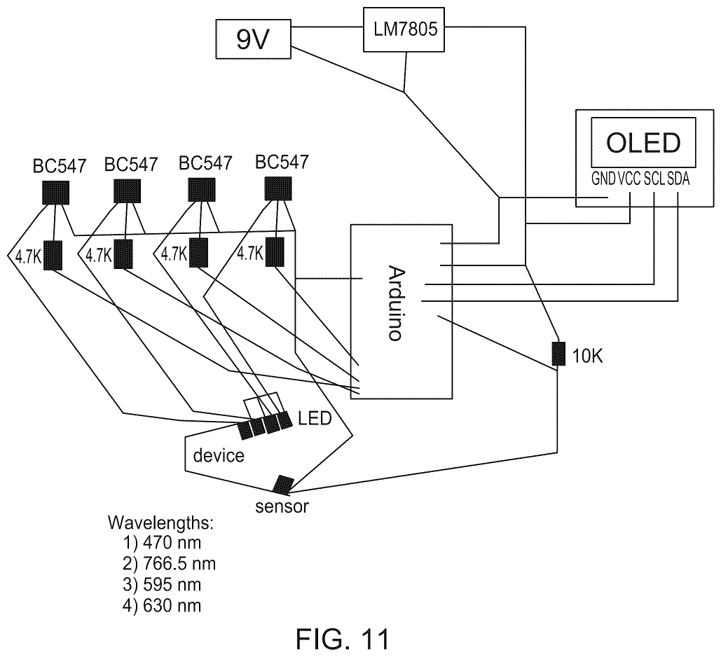

An exemplary device for estimating glucose levels from electrolyte absorbance measurements was designed and fabricated to include a i) light emitting unit (900) comprising light sources (901, 902, 903) configured to emit light at multiple wavelengths (e.g., including the three distinct wavelengths identified in Example 1); and ii) a light sensing unit (904) configured to detect the emitted light with a photodetector, e.g., photodiode sensor (906), as shown in FIG. 9A. A microcontroller (908) was included to manage light output and process light absorbance measurements. FIG. 8 is a schematic diagram of an exemplary circuit of the microcontroller (908) shown in FIGS. 9A and 9B. FIG. 11 shows a second illustrative circuit diagram for embodiments comprising the three light sources of FIG. 8 and an additional 650 nm light source

More specifically, and as shown in FIGS. 9A and 9B, the exemplary device designs included a processor microcontroller (908), a light emitting unit (900) contained at least partially within first housing (910) and having an LED array (901, 902, 903), a light sensing unit (904) including a photodetector, e.g., photodiode sensor (906) contained at least partially within second housing (912), a 3D-printed clamp (930) including the first housing (910) coupled to the second housing (912) via a hinge mechanism (914) and configured to grip a portion of the body having a blood sample, e.g., a finger, via springs (916), a power source (e.g., a battery (918)), a battery holder including a clip (920), and an electronics cover (928). The LED array (901, 902, 903) contained three LED sources corresponding to useful wavelengths for detection of potassium, sodium, and phosphate as identified in Example 1 (766.5 nm, 595 nm, and 470 nm, respectively). Alternate embodiments without the 470 nm LED or including a display (932 in FIG. 10A) are also contemplated. The three LED sources of the array (901, 902, 903) were positioned within openings (924) in the first housing (910), as shown in FIGS. 13A-B. The circuit components included wires [AWG=20], transistors, resistors, and a connector. The 3D-printed clamp (930) in the closed configuration had a sample opening (926) configured for finger or other sample placement, and a connector (922) configured to join the power source, a 9V battery (918), to the clamp. The difference between the designs in FIGS. 9A and 9B is that in FIG. 9B, the openings (924) for the LED sources (901, 902, 903) are arranged linearly in the first housing (910), which may be beneficial for taking measurements from the ear or lip, and a larger sample opening (926) in order to better accommodate tissue of the ear or lip. In FIGS. 13A and 13B, an enlarged view of the first housing of FIG. 9B (see FIG. 13A) and the second housing of FIG. 9B (see FIG. 13B) is provided that shows the configuration of the inner surface of the housings. More specifically, in FIG. 13A, first housing (910) includes three openings (924) in which three LED sources may be placed. Additionally, the inner surface (934) of the first housing (910) may include a first elongate recess (936) extending along a midline thereof and configured to help hold a body part (or tissue of the body part) between the first and second housings when the clamp is in a closed configuration. The second housing (912) shown in FIG. 13B may include an opening (938) in which the photodiode sensor (906) may be placed. The inner surface (940) of the second housing (912) may also include a second elongate recess (942) extending along a midline thereof and configured to work with the first elongate recess (936) to help hold a body part (or tissue of the body part) between the first and second housings when the clamp is in a closed configuration.

A prototype was then fabricated according to the exemplary system and device designs, as shown in FIG. 10A and FIG. 10B.

### NUMBERED EMBODIMENTS OF THE DISCLOSURE

Notwithstanding the appended claims, the disclosure sets forth the following numbered embodiments:

### Embodiment Set Number One

1. A method for non-invasively quantifying blood glucose level of a subject animal, the method comprising the steps of:
a) optically measuring concentration of at least two of the following:
   i) potassium concentration in the subject's circulating blood;
   ii) sodium concentration in the subject's circulating blood;
   iii) phosphate concentration in the subject's circulating blood; and
b) determining glucose content estimates by:
   i) independently multiplying the potassium, sodium, and/or phosphate concentrations measured in step (a) by its respective glucose modification factor; and
c) reconciling the glucose content estimates from step (b), thereby quantifying blood glucose level of a subject animal.
1.1 The method of embodiment 1, wherein step (a) involves optically measuring potassium, sodium, and phosphate concentrations.
1.2 The method of embodiment 1 or 1.1, wherein the potassium concentration is measured at about 766.5 nm.
1.3 The method of any one of embodiments 1-1.2, wherein the sodium concentration is measured at about 595 nm.
1.4 The method of any one of embodiments 1-1.2, wherein the phosphate concentration is measured at about 470 nm.
1.5 The method of any one of embodiments 1.2-1.4, wherein concentrations of potassium, sodium, and/or phosphate are calculated using the Beer-Lambert equation.
1.6 The method of any one of embodiments 1-1.5, wherein the method further comprises optically measuring blood volume of the circulating blood.
1.7 The method of embodiment 1.6, wherein blood volume is measured at about 650 nm.
1.8 The method of any one of embodiments 1.6 or 1.7, wherein the concentrations of potassium, sodium, and/or phosphate measured in step (a) are normalized based on the measured blood volume.
2. A method for non-invasively quantifying blood glucose level of a subject animal, the method comprising the steps of:
a) measuring, via a photodetector, absorbance from of the subject's circulating blood from a light source at least two of the following wave lengths:
   i) about 766.5 nm;
   ii) about 595 nm;
   iii) about 470 nm; and
b) determining glucose content estimates of absorbance measurements from (a) by:
   i) dividing the absorbance measured in (a)(i) by about 1.247×Lᵢ) and multiplying that number by a predetermined glucose modification factor for absorbance 766.5 nm;
   ii) dividing the absorbance measured in (a)(ii) by about (3.727×Lᵢᵢ) and multiplying that number by a predetermined glucose modification factor for absorbance 595 nm;
   iii) dividing the absorbance measured in (a)(iii) by about (3.533×Lᵢᵢᵢ); and multiplying that number by a predetermined glucose modification factor for absorbance 470 nm; and
c) reconciling the glucose content estimates from step (b), thereby quantifying blood glucose level of a subject animal;
wherein L₍ᵢ₋ᵢᵢᵢ₎ is distance each light travels though the subject's circulating blood in each of elements (a)(i)-(iii), respectively.
2.1 The method of embodiment 1, wherein step (a) involves measuring at all three wavelengths recited in step (a)(i)-(iii).
2.2 The method of any one of embodiments 2-2.1, wherein step (a) further comprises optically determining blood volume of circulating blood.
2.3 The method of embodiment 2.2, wherein blood volume is determined by measuring, via a photodetector, absorbance from of the subject's circulating blood from a light source of about 650 nm.
2.4 The method of any one of embodiments 2.2 or 2.3, wherein L₍ᵢ₋ᵢᵢᵢ₎ is based on the measured blood volume.
3. An apparatus for non-invasively quantifying blood glucose level of a subject animal, the apparatus comprising:
a) two more light sources selected from the group consisting of:
   i) a light source capable of emitting light at about 766.5 nm;
   ii) a light source capable of emitting light at about 595 nm;
   iii) a light source capable of emitting light at 470 nm;
b) a photodetector, capable of measuring light transmittance at each of the wavelengths of (a)(i)-(iii) present in the apparatus; the photodetector positioned so as to detect light transmitted through an area of blood flow by the light sources;
c) a processor, operably linked to the photodetector and light sources; the processor configured to:
   i) determine glucose content estimates by:
      1) dividing the log 10 transmittance measured by the photodetector of light emitted by the light source (a)(i) by ([extinction coefficient of potassium]×Lᵢ); and multiplying that number by a predetermined glucose modification factor for absorbance 766.5 nm;
      2) dividing the absorbance measured by the photodetector of light emitted by the light source (a)(ii) by ([extinction coefficient of sodium]×Lᵢᵢ); and multiplying that number by a predetermined glucose modification factor for absorbance 595 nm; and
      3) dividing the absorbance measured by the photodetector of light emitted by the light source (a)(iii) by ([extinction coefficient of phosphate]×Lᵢᵢᵢ); and multiplying that number by a predetermined glucose modification factor for absorbance 470 nm; and
   ii) reconcile the glucose content estimates from (c)(i), thereby quantifying blood glucose level of a subject animal; and
d) a power source; and
e) a display;
wherein L₍ᵢ₋ᵢᵢᵢ₎ is distance each light travels though the area of blood flow by the photosensor of element (b).
3.1 The apparatus of embodiment 3, comprising all three light sources recited in (a)(i)-(iii).
3.2 The apparatus of embodiments 3 or 3.1, further comprising a light source capable of emitting light at about 650 nm.
3.3 The apparatus of embodiment 3.2, wherein each of L(a-c) are further normalized based on absorbance at 650 nm measured by the photodetector.
3.4 The apparatus of any one of embodiments 3-3.3, wherein at least one of the light sources is a narrow-band emission light source.
3.5 The apparatus of any one of embodiments 3-3.4, wherein each of the light sources capable of emitting light at about 766.5 nm, about 595 nm, and about 470 nm are narrow-band emission light sources.
3.6 The apparatus of any one of embodiments 3.1-3.4, wherein each of the light sources capable of emitting light at about 766.5 nm, about 595 nm, about 470 nm, and about 650 nm are narrow-band emission light sources.
3.7 The apparatus of any one of embodiments 3-3.6, wherein at least one of the light sources is an light emitting diode (LED).
3.8 The apparatus of any one of embodiments 3-3.6, wherein each of the light sources capable of emitting light at about 766.5 nm, about 595 nm, and about 470 nm are LED.
3.9 The apparatus of any one of embodiments 3.2-3.6, wherein each of the light sources capable of emitting light at about 766.5 nm, about 595 nm, about 470 nm, and about 650 nm are LED.
4. An apparatus for non-invasively quantifying blood glucose level of a subject animal, the apparatus comprising:
a) gripping means for releasably gripping a body part, the gripping means comprising first and second housings interconnected by a pivot means which allows the first and second housings to pivot relative to one another to releasably grip the body part inserted between the first and second housings, the first and second housings being in electrical communication with each other;
b) a light emitting unit comprised within the first housing; the light emitting unit positioned so as to direct light towards the second housing, through a body part gripped between the first and second housings, the light emitting unit comprising at least two of:
   i) a light source capable of emitting light at about 766.5 nm;
   ii) a light source capable of emitting light at about 595 nm;
   iii) a light source capable of emitting light at 470 nm;
c) a light sensing unit, comprised within the second housing; the light sensing unit comprising a photodetector capable of measuring light transmittance from the light emitting unit, through the gripped body part, at each of the wavelengths produced by the light emitting unit.
4.1 The apparatus of embodiment 4, further comprising a means for determining the distance between the first housing and the second housing.
4.2. The apparatus of embodiment 4 or 4.1, wherein the pivot means is capable of measuring an angle between the first and second housings, thereby measuring the distance between the first housing and the second housing.
4.3 The apparatus of any one of embodiments 4-4.2, wherein the light emitting unit comprises all three light sources recited in (b)(i)-(iii).
4.4 The apparatus of any one of embodiments 4-4.3, further comprising a light source capable of emitting light at about 650 nm.
4.5 The apparatus of any one of embodiments 4-4.4, wherein at least one of the light sources is a narrow-band emission light source.
4.6 The apparatus of any one of embodiments 4-4.5, wherein each of the light sources capable of emitting light at about 766.5 nm, about 595 nm, and about 470 nm are narrow-band emission light sources.
4.7 The apparatus of any one of embodiments 4.4-4.5, wherein each of the light sources capable of emitting light at about 766.5 nm, about 595 nm, about 470 nm, and about 650 nm are narrow-band emission light sources.
4.8 The apparatus of any one of embodiments 4-4.7, wherein at least one of the light sources is an light emitting diode (LED).
4.9 The apparatus of any one of embodiments 4-4.7, wherein each of the light sources capable of emitting light at about 766.5 nm, about 595 nm, and about 470 nm are LED.
4.11 The apparatus of any one of embodiments 4.4-4.5, wherein each of the light sources capable of emitting light at about 766.5 nm, about 595 nm, about 470 nm, and about 650 nm are LED.
4.12 The apparatus of any one of embodiments 4-4.11, further comprising d) a power source.
4.13 The apparatus of any one of embodiments 4-4.12, further comprising e) a display.
4.14 The apparatus of any one of embodiments 4-4.13, wherein the light sensing unit comprises a plurality of photodetectors.
4.15 The apparatus of embodiment 4.15, wherein the light sensing unit comprises at least 2, 3, 5, 6, 7, or 8 photodetectors.
4.16 The apparatus of embodiments 4.14 or 4.15, wherein each photodetector is arranged along the second housing at an angle of least 5 degrees apart from each other, the angle measured from the light emitting unit, through the body part, and to the photodetector.
4.17 The apparatus of any one of embodiments 4-4.16, wherein the apparatus comprises a memory unity, capable of storing a first data matrix of photo measurements gathered by the photodetector.
4.18 The apparatus of embodiment 4.17, wherein the apparatus comprises a processor configured to convert the measurements in the first data matrix into a glucose concentration, thereby quantifying glucose in a body port.
4.19 The apparatus of any one of embodiments 4-4.18, wherein the body part is selected from the group consisting of: a finger, an ear lobe, a lip, a nose, and a toe.
5. A method for non-invasively quantifying glucose level of blood, the method comprising the steps of:
a) measuring, via a photodetector, absorbance from blood from a light source at least two of the following wave lengths:
   i) about 766.5 nm;
   ii) about 595 nm;
   iii) about 470 nm; and
b) determining glucose content estimates of absorbance measurements from (a) by:
   i) dividing the absorbance measured in (a)(i) by about ([extinction coefficient of potassium]×Lᵢ);
   ii) dividing the absorbance measured in (a)(ii) by about ([extinction coefficient of sodium]×Lᵢᵢ);
   iii) dividing the absorbance measured in (a)(iii) by about ([extinction coefficient of phosphate]×Lᵢᵢᵢ); and
   iv) adjusting the values of step (b)(i)-(iii) via a regression model, thereby producing a glucose content estimate; and
c) reconciling the glucose content estimates from step (b), thereby quantifying blood glucose level of a subject animal;
   wherein L₍ᵢ₋ᵢᵢᵢ₎ is distance each light travels though the blood in each of elements (a)(i)-(iii), respectively.

### Embodiment Set Number Two

1. A method for non-invasively quantifying blood glucose level of a subject, the method comprising the steps of:
a) irradiating a sample site of the subject with a light source at least at one of (i) about 766.5 nm, (ii) about 595 nm, and (iii) about 470 nm;
b) measuring an absorbance value from the the light source(s) (i)-(iii) through the sample site;
c) determining glucose content estimates by at least one of (cᵢ)-(cᵢᵢᵢ):
   i) dividing the absorbance measured in (aᵢ) by (about 1.247×L);
   ii) dividing the absorbance measured in (aᵢᵢ) by (about 3.727×L);
   iii) dividing the absorbance measured in (aᵢᵢᵢ) by (about 3.533×L);
   wherein L is a distance that light traveled through the sample site in a); and
d) applying a regression model to the glucose content estimates from each of steps (cᵢ-cᵢᵢᵢ) that were determined, thereby quantifying blood glucose level of a subject animal.
1.1 The method of embodiment 1, wherein the method further comprises step e) reconciling glucose content estimates to quantify blood glucose of the subject.
2. The method of embodiment 1, wherein the irradiating a sample site of the subject with a light source occurs at:
   about 766.5 nm, and about 595 nm;
   about 766.5 nm, and about 470 nm;
   about 595 nm, and about 470 nm; or
   about 766.5 nm, about 595 nm, and about 470 nm.
3. The method of embodiment 1, wherein the irradiating a sample site of the subject with a light source occurs at about 766.5 nm, about 595 nm, and about 470 nm.
4. The method of any one of the previous embodiments, wherein the method further comprises measuring the intensity of the light source at each wavelength before irradiating the sample site of the subject.
5. The method of any one of the previous embodiments, wherein the method further comprises measuring L.
6. The method of any one of the previous embodiments, wherein the regression comprises a polynomial regression, a ridge regression, a lasso regression, a decision tree regression, or a random forest regression.
7. The method of any one of the previous embodiments, wherein the sample site is a finger, toe, lip, ear, or tongue.
8. The method of any one of the previous embodiments, wherein the sample site is a finger.
9. The method of any one of the previous embodiments, wherein the subject is a human.
10. An apparatus for non-invasively quantifying blood glucose level of a subject, the apparatus comprising:
a) a light source capable of irradiating a sample site at least at one of (i) about 766.5 nm, (ii) about 595 nm, and (iii) about 470 nm;
b) a sensor, capable of measuring light transmittance at each of the wavelengths of (aᵢ)-(aᵢᵢᵢ); the sensor positioned so as to detect light transmitted through a sample site by the light source of (a);
c) a processor, operably linked to the sensor and light sources; the processor configured to:
   i) determining glucose content estimates by:
      1) dividing the absorbance measured in (aᵢ) by (about 1.247×L);
      2) dividing the absorbance measured in (aᵢᵢ) by (about 3.727×L);
      3) dividing the absorbance measured in (aᵢᵢᵢ) by (about 3.533×L);
      wherein L is a distance that light traveled through the sample site in a); and
   ii) correlating the glucose content estimates from step (d), thereby quantifying blood glucose level of a subject.
11. The apparatus of embodiment 10, wherein the irradiating a sample site of the subject with a light source occurs at:
   about 766.5 nm, and about 595 nm;
   about 766.5 nm, and about 470 nm;
   about 595 nm, and about 470 nm; or
   about 766.5 nm, about 595 nm, and about 470 nm.
12. The apparatus of embodiment 10, wherein the irradiating a sample site of the subject with a light source occurs at about 766.5 nm, about 595 nm, and about 470 nm.
12.1. The apparatus of any one of the previous embodiments, wherein the absorbance is determined by dividing the log 10 transmittance measured by light sensor of each wavelength of light emitted by each light source at each specific wavelength.
13. The apparatus of any one of the previous embodiments, wherein the apparatus comprises a module for measuring or calculating the intensity of the light source at each wavelength use to irradiate the sample site of the subject.
14. The apparatus of any one of the previous embodiments, wherein the apparatus comprises a module for determining or measuring L.
15. The apparatus of any one of the previous embodiments, wherein the correlation comprises applying a polynomial regression, a ridge regression, a lasso regression, a decision tree regression, or a random forest regression.
16. The apparatus of any one of the previous embodiments, wherein the sample site is a finger, toe, lip, ear, or tongue.
17. The apparatus of any one of the previous embodiments, wherein the sample site is a finger.
18. The apparatus of any one of the previous embodiments, wherein the subject is a human.
19. A method for quantifying blood glucose levels, the method comprising the steps of:
   a) irradiating a sample with a light source at least at one of (i) about 766.5 nm, (ii) about 595 nm, and (iii) about 470 nm;
   b) measuring an absorbance value from the the light source(s) (i)-(iii) through the sample;
   c) determining glucose content estimates by at least one of (cᵢ)-(cᵢᵢᵢ):
      i) dividing the absorbance measured in (aᵢ) by (about 1.247×L);
      ii) dividing the absorbance measured in (aᵢᵢ) by (about 3.727×L);
      iii) dividing the absorbance measured in (aᵢᵢᵢ) by (about 3.533×L);
      wherein L is a distance that light traveled through the sample site in a); and
   d) applying a regression model to the glucose content estimates from each of steps (cᵢ-cᵢᵢᵢ) that were determined, thereby quantifying blood glucose level.

### Embodiment Set Number 3

1. An apparatus for quantifying blood glucose, the apparatus comprising:
a) a gripping means for releasably gripping a blood sample, the gripping means comprising first and second housings interconnected by a pivot means which allows the first and second housings to pivot relative to one another to releasably grip the blood sample inserted between the first and second housings, the first and second housings being in electrical communication with each other;
b) a light emitting unit comprised within the first housing; the light emitting unit positioned so as to direct light towards the second housing, through the blood sample gripped between the first and second housings, the light emitting unit comprising at least two of:
   i) a light source capable of emitting light at 740-780 nm;
   ii) a light source capable of emitting light at 580-620 nm;
   iii) a light source capable of emitting light at 450-490 nm; and
c) a light sensing unit comprised within the second housing; the light sensing unit comprising a photodetector capable of measuring light transmittance from the light emitting unit, through the gripped blood sample.
2. The apparatus of embodiment 1, further comprising a means for determining the distance between the light emitting unit and the light sensing unit.
3. The apparatus of embodiment 1 or 2, wherein the pivot means is capable of measuring an angle between the first and second housings, thereby determining the light emitting unit and the light sensing unit.
4. The apparatus of any one of embodiments 1-3, wherein the light emitting unit comprises all three light sources recited in (b)(i)-(iii).
5. The apparatus of any one of embodiments 1-4, wherein the light emitting unit further comprises a light source capable of emitting light at 625-675 nm.
6. The apparatus of any one of embodiments 1-5, wherein at least one of the light sources is a narrow-band emission light source.
7. The apparatus of any one of embodiments 1-6, wherein each of the light sources capable of emitting light at 740-780 nm, 580-620 nm, and 450-490 nm are narrow-band emission light sources.
8. The apparatus of any one of embodiments 1-7, wherein each of the light sources capable of emitting light at 740-780 nm, 580-620 nm, 450-490 nm, and 625-675 nm are narrow-band emission light sources.
9. The apparatus of any one of embodiments 1-8, wherein at least one of the light sources is a light emitting diode (LED).
10. The apparatus of any one of embodiments 1-9, wherein:
   light source (b)(i) is capable of emitting light at about 766.5 nm;
   light source (b)(ii) is capable of emitting light at about 595 nm; and
   light source (b)(iii) is capable of emitting light at about 470 nm.
11. The apparatus of embodiment 10, wherein each of the light sources capable of emitting light at about 766.5 nm, about 595 nm, and about 470 nm are LED.
12. The apparatus of any one of embodiments 1-11, further comprising d) a power source.
13. The apparatus of any one of embodiments 1-12, further comprising e) a display.
14. The apparatus of any one of embodiments 1-13, wherein the light sensing unit comprises a plurality of photodetectors.
15. The apparatus of embodiment 14, wherein each photodetector is arranged along the second housing at an angle of at least 5 degrees apart from each other, the angle measured from the light emitting unit, through the blood sample, and to the photodetector.
16. The apparatus of any one of embodiments 1-15, wherein the apparatus comprises a memory unity, capable of storing a first data matrix of photo measurements gathered by the photodetector.
17. The apparatus of embodiment 16, wherein the apparatus comprises a processor configured to convert the measurements in the first data matrix into a glucose concentration, thereby quantifying glucose in the blood sample.
18. A method for quantifying blood glucose level, the method comprising the steps of:
a) measuring, via a photodetector, absorbance of blood from a light within at least two of the following wavelength ranges:
   i) 740-780 nm;
   ii) 580-620 nm;
   iii) 450-490 nm; and
b) determining glucose content estimates from the absorbance measurements from (a) by:
   i) dividing the absorbance measured in (a)(i) by (about 1.247×Lᵢ);
   ii) dividing the absorbance measured in (a)(ii) by (about 3.727×Lᵢᵢ);
   iii) dividing the absorbance measured in (a)(iii) by (about 3.533×Lᵢᵢᵢ); and
   iv) separately applying a regression model to the values of step (b)(i)-(iii), thereby producing a plurality of glucose content estimates; and
c) reconciling the glucose content estimates from step (b), thereby quantifying blood glucose level of the blood;
wherein L₍ᵢ₋ᵢᵢᵢ₎ is distance each light travels though the blood in each of elements (a)(i)-(iii), respectively.
18.1 A method for quantifying blood glucose level, the method comprising the steps of:
a) measuring, via a photodetector, absorbance of blood from a light within at least two of the following wavelength ranges:
   i) 740-780 nm;
   ii) 580-620 nm;
   iii) 450-490 nm; and
b) determining glucose content estimates from the absorbance measurements from (a) by applying Beer's Law to:
   i) absorbance measured in (a)(i) for potassium;
   ii) absorbance measured in (a)(ii) for phosphate;
   iii) absorbance measured in (a)(iii) for sodium; and
c) applying a regression model to the values of step (b)(i)-(iii), thereby producing a plurality of glucose content estimates; and
d) reconciling the glucose content estimates from step (c), thereby quantifying blood glucose level of the blood.
19. The method of embodiment 18 or 18.1, comprising irradiating the blood with light wavelengths of about 766.5 nm for a(i), about 595 nm for a(ii), and about 470 nm for a(iii), if measured.
20. The method of any one of embodiments 18-19, wherein step (c) and/or (d) comprises executing a neural network or machine learning algorithm.

### Embodiment Set Number 4

1. A system for measuring glucose concentrations comprising:
a) a clamp configured to attach to a body site, the clamp comprising a first housing coupled to a second housing, wherein the first housing comprises a light emitting unit, the light emitting unit comprising at least two light sources, and wherein the second housing comprises one or more photodetectors; and
b) a processor configured to receive data from the one or more photodetectors indicative of a concentration of one or more electrolytes in a fluid at the body site and determine a glucose concentration of the fluid based on the concentration of the one or more electrolytes.
2. The system of embodiment 1, wherein the first housing has a length between about 2.0 cm to about 10 cm.
4. The system of embodiment 1, wherein the first housing has a width between about 2.0 cm to about 6.0 cm.
5. The system of embodiment 1, wherein the second housing has a length between about 2.0 cm to about 10 cm.
6. The system of embodiment 1, wherein the second housing has a width between about 2.0 cm to about 6.0 cm.
7. The system of embodiment 1, wherein a length and a width of the first and second housing is the same.
8. The system of embodiment 1, wherein the first and second housings comprise a polymeric material.
9. The system of embodiment 8, wherein the polymeric material is ABS (acrylonitrile butadiene styrene), polycarbonate, polyethylene, polypropylene, or combinations thereof.
10. The system of embodiment 1, wherein the first and second housings comprise a metal or a metal alloy.
11. The system of embodiment 10, wherein the metal is stainless steel, aluminum, nickel, or combinations thereof.
12. The system of embodiment 1, wherein the first housing is coupled to the second housing via a hinge.
13. The system of embodiment 12, wherein the hinge is a butt hinge, pivot hinge, or an integral hinge.
14. The system of embodiment 13, wherein the hinge transitions the clamp from a closed configuration to an open configuration.
15. The system of embodiment 1, wherein the at least two light sources comprise a light-emitting diode (LED).
16. The system of embodiment 1, wherein the light emitting unit is configured to emit light at a wavelength between about 740 nm to about 780 nm.
17. The system of embodiment 1, wherein the light emitting unit is configured to emit light at a wavelength between about 580 nm to about 620 nm.
18. The system of embodiment 1, wherein the light emitting unit is configured to emit light at a wavelength between about 450 nm to about 490 nm.
19. The system of embodiment 1, wherein the light emitting unit is configured to emit light at a wavelength between about 625 nm to about 675 nm.
20. The system of embodiment 1, wherein the one or more photodetectors comprise a photodiode.
21. The embodiment of claim 1, wherein the one or more processors is configured to apply a glucose modification factor to the received data from the one or more photodetectors indicative of the concentration of one or more electrolytes to determine the glucose concentration.
22. The embodiment of claim 1, wherein the one or more processors is configured to apply a machine learning algorithm to the received data from the one or more photodetectors indicative of the concentration of one or more electrolytes to determine the glucose concentration.
23. The system of embodiment 22, wherein the machine learning algorithm comprises a random forest, a boosted decision tree, a classification tree, a regression tree, a bagging tree, a neural network, a rotation forest, or combinations thereof.
24. The system of embodiment 23, wherein the neural network comprises a convolutional neural network (CNN), a deep convolutional neural network, a recurrent neural network (RNN), or combinations thereof.
25. The system of embodiment 1, wherein the one or more electrolytes is sodium, potassium, or phosphate.
26. The system of embodiment 1, wherein at least a portion of the clamp is disposable.
27. The system of embodiment 1, wherein an inner surface of at least one or the first and second housings comprises a conformable material.
28. The system of embodiment 27, wherein the conformable material comprises silicone.
29. The system of embodiment 1, wherein the body site is a finger, toe, ear, lip, tongue, arm, wrist, or ankle.
30. The system of embodiment 1, wherein the fluid is blood, plasma, urine, cerebrospinal fluid, pleural fluid, ascitic fluid, or sweat.
31. The system of embodiment 30, wherein the fluid is blood.
32. The system of embodiment 1, further comprising a display.
33. The system of embodiment 1, further comprising a user interface.
34. The system of embodiment 1, further comprising a power source.
35. The system of embodiment 34, wherein the power source comprises a rechargeable or non-rechargeable battery.
36. The system of embodiment 34, wherein the power source is removably secured to the first housing or the second housing.
37. A method for measuring glucose concentrations comprising:
a) attaching a clamp to a body site, the clamp comprising at least two light sources and one or more photodetectors;
b) irradiating the body site with light wavelengths associated with two or more electrolytes, wherein the two or more electrolytes are sodium, potassium, or phosphate;
c) receiving data from the one or more photodetectors indicative of a concentration of one or more electrolytes in a fluid at the body site; and
d) determining a glucose concentration of the fluid based on the concentration of the one or more electrolytes.
38. The method of embodiment 37, wherein the light wavelengths are associated with sodium and potassium.
39. The method of embodiment 37, wherein the light wavelengths are associated with sodium and phosphate.
40. The method of embodiment 37, wherein the light wavelengths are associated with potassium and phosphate.
41. The method of embodiment 37, wherein the light wavelengths associated with sodium range between about 580 nm to about 620 nm.
42. The method of embodiment 37, wherein the light wavelengths associated with potassium range between about 740 nm to about 780 nm.
43. The method of embodiment 37, wherein the light wavelengths associated with phosphate range between about 450 nm to about 490 nm.
44. The method of embodiment 37, further comprising using one or processors to receive the data from the one or more photodetectors indicative of a concentration of the one or more electrolytes in the fluid at the body site.
45. The method of embodiment 44, the one or more processors is configured to apply a machine learning algorithm to the received data from the one or more photodetectors indicative of the concentration of one or more electrolytes to determine the glucose concentration.
46. The method of embodiment 45, wherein the machine learning algorithm comprises a random forest, a boosted decision tree, a classification tree, a regression tree, a bagging tree, a neural network, a rotation forest, or combinations thereof.
47. The method of embodiment 46, wherein the neural network comprises a convolutional neural network (CNN), a deep convolutional neural network, a recurrent neural network (RNN), or combinations thereof.
48. The method of embodiment 37, wherein the body site is a finger, toe, ear, lip, tongue, arm, wrist, or ankle.
49. The method of embodiment 37, wherein the fluid is blood, plasma, urine, cerebrospinal fluid, pleural fluid, ascitic fluid, and sweat.
50. The method of embodiment 49, wherein the fluid is blood.
51. A system for measuring glucose concentrations comprising:
a) a container comprising a sample;
b) a clamp configured to attach to the container, the clamp comprising a first housing coupled to a second housing, wherein the first housing comprises a light emitting unit, the light emitting unit comprising at least two light sources, and wherein the second housing comprises one or more photodetectors; and
c) a processor configured to receive data from the one or more photodetectors indicative of a concentration of one or more electrolytes in the sample and determine a glucose concentration of the sample based on the concentration of the one or more electrolytes.
52. The system of embodiment 51, wherein the sample is a blood sample.
53. A method for measuring glucose concentrations comprising:
a) attaching a clamp to a container comprising a sample, the clamp comprising at least two light sources and one or more photodetectors;
b) irradiating the sample with light wavelengths associated with two or more electrolytes, wherein the two or more electrolytes are sodium, potassium, or phosphate;
c) receiving data from the one or more photodetectors indicative of a concentration of one or more electrolytes in the sample; and
d) determining a glucose concentration of the sample based on the concentration of the one or more electrolytes.
54. The method of embodiment 53, wherein the sample is a blood sample.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

1. An apparatus for quantifying blood glucose, the apparatus comprising:
a) a grip for releasably gripping a blood sample, the grip comprising first and second housings interconnected by a pivot configured to position the first and second housings relative to one another to releasably grip the blood sample inserted between the first and second housings, the first and second housings being in electrical communication with each other;
b) a light emitting unit comprised at least partially within the first housing; the light emitting unit positioned so as to direct light towards the second housing, through the blood sample gripped between the first and second housings, the light emitting unit comprising at least two of:
i) a light source capable of emitting light at 740-780 nm;
ii) a light source capable of emitting light at 580-620 nm; and
iii) a light source capable of emitting light at 450-490 nm;
c) a light sensing unit comprised at least partially within the second housing; the light sensing unit comprising a photodetector capable of measuring light transmittance from the light emitting unit, through the gripped blood sample; and
d) a processor configured to receive data from the photodetector indicative of a concentration of one or more electrolytes in the blood sample and determine a glucose concentration of the blood sample based on the concentration of the one or more electrolytes,
wherein the one or more electrolytes comprise sodium, potassium, phosphate, or a combination thereof.

2. The apparatus of claim 1, wherein the processor is configured to apply a glucose modification factor to the received data from the photodetector indicative of the concentration of one or more electrolytes to determine the glucose concentration.

3. The apparatus of claim 1, wherein the processor is configured to apply a machine learning algorithm to the received data from photodetector indicative of the concentration of one or more electrolytes to determine the glucose concentration.

4. The apparatus of claim 1, further comprising a mechanism for determining the distance between the light emitting unit and the light sensing unit.

5. The apparatus of claim 1, wherein the pivot is configured to measure an angle between the first and second housings, thereby determining the position of the light emitting unit and the light sensing unit.

6. The apparatus of claim 1, wherein the light emitting unit comprises all three light sources recited in (b)(i)-(iii).

7. The apparatus of claim 1, wherein the light emitting unit further comprises a light source configured to emit light at about 625-675 nm.

8. The apparatus of claim 1, wherein at least one of the light sources is a narrow-band emission light source.

9. The apparatus of claim 6, wherein each of the light sources is a narrow-band emission light source.

10. The apparatus of claim 7, wherein the light emitting unit comprises all three light sources recited in (b)(i)-(iii), and wherein each of the light sources is a narrow-band emission light source.

11. The apparatus of claim 1, wherein at least one of the light sources is a light emitting diode (LED).

12. The apparatus of claim 1, wherein:
light source (b)(i) is configured to emit light at about 766.5 nm;
light source (b)(ii) is configured to emit light at about 595 nm; and
light source (b)(iii) is configured to emit light at about 470 nm.

13. The apparatus of claim 12, wherein each of the light sources comprises an LED configured to emit light at about 766.5 nm, about 595 nm, or about 470 nm.

14. The apparatus of claim 1, further comprising e) a power source.

15. The apparatus of claim 1, further comprising f) a display.

16. The apparatus of claim 1, wherein the light sensing unit comprises a plurality of photodetectors.

17. The apparatus of claim 1, wherein each photodetector is arranged along the second housing at an angle of at least 5 degrees apart from each other, the angle measured from the light emitting unit, through the blood sample, and to the photodetector.

18. The apparatus of claim 1, wherein the apparatus comprises a memory unity configured to store a first data matrix of photo measurements gathered by the photodetector.

19. The apparatus of claim 18, wherein the apparatus comprises a processor configured to convert the measurements in the first data matrix into a glucose concentration, thereby quantifying glucose in the blood sample.

20. A method for quantifying blood glucose level, the method comprising the steps of:
a) measuring, via a photodetector, absorbance of blood from a light within at least two of the following wavelength ranges:
i) 740-780 nm;
ii) 580-620 nm;
iii) 450-490 nm; and
b) determining glucose content estimates from the absorbance measurements from (a) by:
i) dividing the absorbance measured in (a)(i) by (about 1.247×Lᵢ);
ii) dividing the absorbance measured in (a)(ii) by (about 3.727×Lᵢᵢ);
iii) dividing the absorbance measured in (a)(iii) by (about 3.533×Lᵢᵢᵢ); and
iv) separately applying a regression model to the values of step (b)(i)-(iii), thereby producing a plurality of glucose content estimates; and
c) reconciling the glucose content estimates from step (b), thereby quantifying blood glucose level of the blood,
wherein L₍ᵢ₋ᵢᵢᵢ₎ is distance each light travels though the blood in each of elements (a)(i)-(iii), respectively.
